(19) United States
(12) Patent Application Publication
(10) Pub. No.: US 2017/0058045 A1
(43) Pub. Date: Mar. 2, 2017

(54) METHODS, COMPOSITIONS AND KITS FOR CELL SPECIFIC MODULATION OF TARGET ANTIGENS

(71) Applicant: Regeneron Pharmaceuticals, Inc., Tarrytown, NY (US)

(72) Inventors: Jesper GROMADA, Scarsdale, NY (US); Eric SMITH, New York, NY (US); Andrew MURPHY, Croton-on-Hudson, NY (US); Nicholas PAPADOPOULOS, LaGrangeville, NY (US); Joel MARTIN, Putnam Valley, NY (US); George YANCOPOULOS, Yorktown Heights, NY (US); Douglas MACDONALD, New York, NY (US); David BUCKLER, Sleepy Hollow, NY (US)

(21) Appl. No.: 15/119,986

(22) PCT Filed: Feb. 20, 2015
(86) PCT No.: PCT/US15/16737

§ 371 Date: Aug. 18, 2016

Related U.S. Application Data
(60) Provisional application No. 61/942,791, filed on Feb. 21, 2014.

(51) Int. Cl. C07K 16/32; A61K 39/395; C07K 16/28

(52) U.S. Cl. CPC C07K 16/32; C07K 16/2803; C07K 16/283; A61K 39/39558; C07K 16/2869; C07K 16/2863; C07K 16/2866; C07K 2317/31; A61K 2039/507

(57) ABSTRACT

The present invention provides, inter alia, a method for cell-specific modulation of a target antigen. The method comprises contacting a target cell having the target antigen on the surface of the target cell with: (a) first multi-specific antigen-binding polypeptide comprising: (i) a cell-specific antigen binding domain (C1), (ii) a target antigen binding domain (T1); and (b) a second multi-specific antigen-binding polypeptide comprising: (i) a cell-specific antigen binding domain (C2), (ii) a target antigen binding domain (T2); wherein C1 and C2 interact with the same cell-specific antigen, and the cell-specific antigen and the target antigen are on the same target cell. Pharmaceutical compositions and kits thereof are also included in the present invention.

### FIELD OF INVENTION

The present invention provides, inter alia, methods for cell-specific modulation of a target antigen. Pharmaceutical compositions and kits for cell-specific modulation of a target antigen are also provided.

### INCORPORATION BY REFERENCE OF SEQUENCE LISTING

This application contains references to amino acids and/or nucleic acid sequences that have been filed concurrently herewith as sequence listing text file T0018P_seq.txt, file size of approximately 874 KB, created on 20 Feb. 2014. The aforementioned sequence listing is hereby incorporated by reference in its entirety pursuant to 37 C.F.R. §1.52(e)(5).

### BACKGROUND OF THE INVENTION

Monoclonal antibody and antibody-derived therapeutics have been developed for the treatment of a variety of diseases. Many of these antibodies are mono-specific and are therefore capable of interacting, activating or interfering with a single target. Efforts have been made to develop antibodies and antibody-derived therapeutics with improved efficacy that have more than one antigen binding specificity, e.g., bi-specific and multi-specific antibodies.

The ability to direct such therapeutic molecules to specific cell types, such as cells of the prostate, breast, kidney, liver, immune system, etc. would be a powerful tool in effecting certain desired outcomes and prevent undesirable side effects. To date, little to no progress has been made in this regard.

There is, inter alia, a need for modulation of proteins in specific cells. The present invention is thus directed to these needs, as well as other related needs.

### SUMMARY OF THE INVENTION

One embodiment of the present invention is a method for cell-specific modulation of a target antigen. The method comprises contacting a target cell having the target antigen on the surface of the target cell with:

(a) a first multi-specific antigen-binding polypeptide comprising:
(i) a cell-specific antigen binding domain (C1), and
(ii) a target antigen binding domain (T1); and

(b) a second multi-specific antigen-binding polypeptide comprising:
(i) a cell-specific antigen binding domain (C2), and
(ii) a target antigen binding domain (T2),
   wherein C1 and C2 interact with the same cell-specific antigen, and the cell-specific antigen and the target antigen are on the same target cell.

Another embodiment of the present invention is a pharmaceutical composition for cell-specific modulation of a target antigen in a subject in need thereof. The pharmaceutical composition comprises a pharmaceutically acceptable diluent or carrier and an effective amount of:

(a) a first multi-specific antigen-binding polypeptide comprising:
(i) a cell-specific antigen binding domain (C1), and
(ii) a target antigen binding domain (T1); and

(b) a second multi-specific antigen-binding polypeptide comprising:
(i) a cell-specific antigen binding domain (C2), and
(ii) a target antigen binding domain (T2),
   wherein C1 and C2 interact with the same cell-specific antigen, and the cell-specific antigen and the target antigen are on the same target cell.

A further embodiment of the present invention is a kit for cell-specific modulation of a target antigen. The kit comprises, packaged together with instructions for their use:

(a) a first multi-specific antigen-binding polypeptide comprising:
(i) a cell-specific antigen binding domain (C1), and
(ii) a target antigen binding domain (T1); and

(b) a second multi-specific antigen-binding polypeptide comprising:
(i) a cell-specific antigen binding domain (C2), and
(ii) a target antigen binding domain (T2),
   wherein C1 and C2 interact with the same cell-specific antigen, and the cell-specific antigen and the target antigen are on the same target cell.

An additional embodiment of the present invention is a method for cell-specific modulation of a target antigen. The method comprises contacting a target cell having the target antigen on the surface of the target cell with:

(a) a first multi-specific antigen-binding polypeptide comprising:
(i) a cell-specific antigen binding domain (C1), and
(ii) a target antigen binding domain (T1);

(b) a second multi-specific antigen-binding polypeptide comprising:
(i) a cell-specific antigen binding domain (C2), and
(ii) a target antigen binding domain (T2),

(c) an antigen-binding polypeptide comprising:
(i) a first cell-specific antigen binding domain (C3), and
(ii) a second cell-specific antigen binding domain (C4),
   wherein the cell-specific antigens and the target antigen are on the same target cell, C1 and C3 interact with a first cell-specific antigen, and C2 and C4 interact with a second cell-specific antigen.

### BRIEF DESCRIPTION OF THE DRAWINGS

FIG. 1A shows a monomeric cell-specific antigen, H, and a homo-dimeric target antigen comprising two monomer A subunits. FIG. 1B shows a multi-specific antigen binding polypeptide, such as a bispecific antibody, comprising a cell-specific antigen binding domain (C1) linked to a target antigen binding domain (T1) and another multi-specific antigen-binding polypeptide, such as a bispecific antibody, comprising a cell-specific antigen binding domain (C2) linked to a target antigen binding domain (T2). C1 and C2 bind to different epitopes on H. T1 and T2 bind to the same epitope on two separate monomers of A. Binding of C1 and C2 to H with simultaneous binding of T1 and T2 to two separate monomers of A facilitates homodimerization of the A monomers. FIG. 1C shows an embodiment similar to that shown in FIG. 1B, except that the target antigen is a heterodimer comprising a subunit A and a subunit B. T1 binds an epitope on target monomer A and T2 binds an epitope on target monomer B. Binding of C1 and C2 to H with simultaneous binding of T1 and T2 to A and B, respectively, facilitates heterodimerization of the A and B subunits.

FIG. 2A shows a monomeric cell-specific antigen, H, and a hetero-trimeric target antigen comprising three subunits: A, B and C. C1 and C2 are cell-specific antigen binding domains specific for two different epitopes on H, while T1 and T2 are target antigen binding domains specific for epitopes on A or B, respectively. Binding of C1 and C2 to H with simultaneous binding of T1 and T2 to A and B, respectively, promotes heterotrimerization of A and B in the presence of C. FIG. 2B shows a homodimeric cell-specific antigen, H comprising two monomer H subunits. C1 and C2 are cell-specific antigen binding domains specific for two different epitopes or the same epitope on H, while T1 and T2 are target antigen binding domains specific for the same epitope on two separate monomers of A. Homodimerization of the two H subunits along with binding of C1 and C2 to the two H monomers and binding of T1 and T2 to the two A monomers, respectively, facilitates homodimerization of the A subunits.

FIG. 3A shows a heterodimeric cell-specific antigen comprising a subunit H1 and a subunit H2 and a homo-dimeric target antigen comprising two monomer A subunits. T1 and T2 are target antigen binding domains specific for the same epitope on two separate monomers of A. Heterodimerization of H1 and H2 along with binding of cell-specific binding domain C1 to H1, cell-specific binding domain C2 to H2, and binding of T1 and T2 to the two A monomers, respectively, facilitates homodimerization of the A subunits. FIG. 3B shows a cell-specific antigen comprising a subunit H which forms a heterotrimer with subunits A and B to form a heterotrimer. Cell-specific binding domains C1 and C2 bind to different epitopes on H, while target binding domains T1 and T2 bind to epitopes A and B, respectively. C1 and C2 binding to H along with simultaneous binding of T1 to A and T2 to B will facilitate heterotrimerization of the H, A, and B subunits.

FIG. 4A shows three different multi-specific antigen binding polypeptides, a cell-specific antigen H1, a separate cell specific antigen H2 that does not complex with H1, and a homodimeric target antigen comprising two monomer A subunits. (1) The first multi-specific antigen binding polypeptide comprises a cell-specific antigen binding domain C1, which binds an epitope on cell specific antigen H1, linked to a target antigen binding domain T1, which binds an epitope on monomer A. (2) The second multi-specific antigen binding polypeptide comprises a cell-specific antigen binding domain C2, which binds an epitope on cell specific antigen H2, linked to a target antigen binding domain T2, which binds an epitope on monomer A. T1 and T2 bind the same epitope on each A monomer. (3) The third multi-specific antigen binding polypeptide comprises a cell-specific antigen binding domain C3, which binds an epitope on cell specific antigen H1, linked to a cell-specific antigen binding domain C4, which binds an epitope on cell specific antigen H2. Binding of C3 and C4 to H1 and H2, thereby bringing H1 and H2 into proximity with each other, along with binding of C1 and C2 to H1 and H2, respectively, and binding of T1 and T2 to the two A monomers, respectively, facilitates homodimerization of A. FIG. 4B shows a monomeric cell-specific antigen, H, and a homo-dimeric target antigen comprising two monomer A subunits, where, instead of facilitating the homodimerization of the target antigen, binding of the multi-specific antigen binding polypeptides to their respective epitopes on A discourages the homodimerization of the target antigen, for example, by keeping the A monomers separated. C1 and C2 are cell-specific antigen binding domains specific for two different epitopes on H. T1 and T2 are target antigen binding domains specific for the same epitope on two separate monomers of A.

FIG. 5A shows a representative multi-specific antigen-binding polypeptide, such as a bispecific antibody, having a cell-specific antigen binding domain (C1), a target antigen binding domain (T1), a first multimerizing domain (M1) and a second multimerizing domain (M2). FIG. 5B shows another multi-specific antigen-binding polypeptide, such as a bispecific antibody, having a cell-specific antigen binding domain (C2), a target antigen binding domain (T2), a first multimerizing domain (M3) and a second multimerizing domain (M4).

### DETAILED DESCRIPTION OF THE INVENTION

Unless defined otherwise, all technical and scientific terms used herein have the same meaning as commonly understood by one of ordinary skill in the art to which this invention belongs.

One embodiment of the present invention is a method for cell-specific modulation of a target antigen. The method comprises contacting a target cell having the target antigen on the surface of the target cell with:

(a) a first multi-specific antigen-binding polypeptide comprising:
(i) a cell-specific antigen binding domain (C1) and
(ii) a target antigen binding domain (T1); and

(b) a second multi-specific antigen-binding polypeptide comprising:
(i) a cell-specific antigen binding domain (C2) and
(ii) a target antigen binding domain (T2),
   wherein C1 and C2 interact with the same cell-specific antigen, and the cell-specific antigen and the target antigen are on the same target cell.

As used herein, the term “modulation” refers to any alteration, variation or change in the properties or activity of the target antigen. For example, modulation, in this context, may include activation or inhibition of a target antigen, such as, e.g., a receptor.

The term “target antigen” refers to any biological molecule expressed on a cell surface that can be bound by an antigen binding polypeptide and that is a suitable target for modulation. Preferably, the target antigens of the present invention are composed of subunits that are activated by multimerization or when clustered together in a cell membrane. More preferably, the “target antigens” of the present invention include, but are not limited to, the FcERI Receptor complex, which includes the FcER1a subunit, the TrkB Receptor, the FGFR1c/FGF21/Beta Klotho complex, the CNTF Receptor complex, which includes the LIFRb and gp130 subunits, the IL-4R/IL-2Rgamma Receptor Complex and the Interferon Gamma Receptor complex, which includes the IFNR1 and IFNR2 subunits

In the context of the present invention, a “cell-specific antigen” refers to any biological molecule that can be bound by an antigen binding polypeptide and which is expressed on the surface of the same cell as the target antigen. Preferably, the “cell specific antigen” is not ubiquitously expressed on all cell types. More preferably, a cell-specific antigen of the present invention is only expressed on the surface of, for example, certain cell types defined by their anatomical origin, location, function, or disease state. For example, the cell-specific antigens of the present invention include, but are not limited to, PD-1, CD300A, Her2, PSMA, KLB, GCGR, and CNTFRa.

In the context of the present invention, a “receptor” refers to any polypeptide, typically expressed on the surface of a cell, that may or may not bind a ligand and that preferably produce downstream effector functions. “Activation” of a given receptor promotes downstream effector functions while “inhibition” of a given receptor discourages such functions. In the present invention, activation and inhibition of a receptor may be ligand-dependent, meaning that said activation and inhibition will not occur unless the receptor's ligand is bound to the receptor. Ligand-independent receptors do not require a bound ligand to be activated or inhibited and are also within the scope of the present invention.

A “target cell” of the present invention may be any cell type, cancerous or non-cancerous that expresses target antigens on its cell surface.

The term “antigen-binding polypeptide,” as used herein, refers to a polypeptide that specifically recognizes an epitope on an antigen, such as a cell-specific antigen and/or a target antigen of the present invention. The term “multi-specific” with reference to an antigen-binding polypeptide means that the polypeptide recognizes different epitopes, either on the same antigen or on different antigens. A multi-specific antigen-binding polypeptide of the present invention can be a single multifunctional polypeptide, or it can be a multimeric complex of two or more polypeptides that are covalently or non-covalently associated with one another. The term “multi-specific antigen-binding polypeptides” includes antibodies or fragments thereof of the present invention that may be linked to or co-expressed with another functional molecule, e.g., another peptide or protein. For example, an antibody or fragment thereof can be functionally linked (e.g., by chemical coupling, genetic fusion, non-covalent association or otherwise) to one or more other molecular entities, such as a protein or fragment thereof to produce a bi-specific or a multi-specific antigen-binding molecule with a second binding specificity.

As used herein, the term “epitope” refers to the portion of the antigen which is recognized by the multi-specific antigen-binding polypeptide. A single antigen (such as an antigenic polypeptide) may have more than one epitope. Epitopes may be defined as structural or functional. Functional epitopes are generally a subset of structural epitopes and are defined as those residues that directly contribute to the affinity of the interaction between the antigen-binding polypeptide and the antigen. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics. Epitopes formed from contiguous amino acids are typically retained on exposure to denaturing solvents, whereas epitopes formed by tertiary folding are typically lost on treatment with denaturing solvents.

The term “domain” refers to any part of a protein or polypeptide having a particular function or structure. Preferably, domains of the present invention bind to cell-specific or target antigens. Cell-specific antigen- or target antigen-binding domains, and the like, as used herein, include any naturally occurring, enzymatically obtainable, synthetic, or genetically engineered polypeptide or glycoprotein that specifically binds an antigen.

The terms “interact” and “bind”, which are used interchangeably herein, mean that two or more molecules form a complex that is relatively stable under physiologic conditions. In the present invention, specific binding may be characterized by an equilibrium dissociation constant (K_{D}) of at least about 10⁻⁵ M, at least about 10⁻⁶ M, at least about 10⁻⁷ M, more preferably at least about 1×10⁻⁸ M, and most preferably at least about 10⁻⁹ M. (e.g., a smaller K_{D} denotes a tighter binding). Methods for determining whether two molecules specifically bind are well known in the art and include, for example, equilibrium dialysis, surface plasmon resonance, and the like.

In one aspect of this embodiment, C1 and C2 bind different epitopes on the cell-specific antigen. In another aspect of this embodiment, T1 and T2 bind the same epitope on the target antigen.

In an additional aspect of this embodiment, the cell-specific antigen is present primarily as a monomer on the cell surface. As used herein, the term “monomer” refers to a polypeptide that does not associate with itself or other polypeptides to form complexes.

In another aspect of this embodiment, the cell-specific antigen is composed of at least two polypeptide subunits on the cell surface. In this embodiment, “on the cell surface” means that the polypeptide subunits are sufficiently accessible to the multi-specific binding polypeptides so that specific binding therebetween may occur. Thus, the specific location of the polypeptide subunits with respect to target cell is not critical, so long as they are accessible to the multispecific binding polypeptide(s) of the present invention. The term “subunit,” as used herein, refers to a polypeptide molecule that can associate with itself or other polypeptide molecules to form a complex. A cell-specific antigen composed of at least two polypeptide subunits on the cell surface may be a dimer, a trimer, a tetramer, or other multimers. Preferably, the cell-specific antigen is a homodimer on the cell surface. As used herein, a “homodimer” means a complex of two of the same polypeptide subunits.

In a further aspect of this embodiment, the target antigen is composed of at least two copies of the same polypeptide subunit on the cell surface. Preferably, the target antigen is a homodimer on the cell surface.

In an additional aspect of this embodiment, the target antigen is composed of at least two different polypeptide subunits on the cell surface. In one preferred embodiment, the target antigen is a heterodimer on the cell surface. As used herein, a “heterodimer” means a complex of two different polypeptide subunits. In another preferred embodiment, the target antigen is a heterotrimer on the cell surface. As used herein, a “heterotrimer” means a complex of three polypeptide subunits, at least two of which are different.

In a further aspect of this embodiment, the first multi-specific antigen-binding polypeptide further comprises:
(iii) a first multimerizing domain (M1) and optionally a second multimerizing domain (M2);
   and the second multi-specific antigen-binding polypeptide further comprises:
(iv) a first multimerizing domain (M3) and optionally a second multimerizing domain (M4).

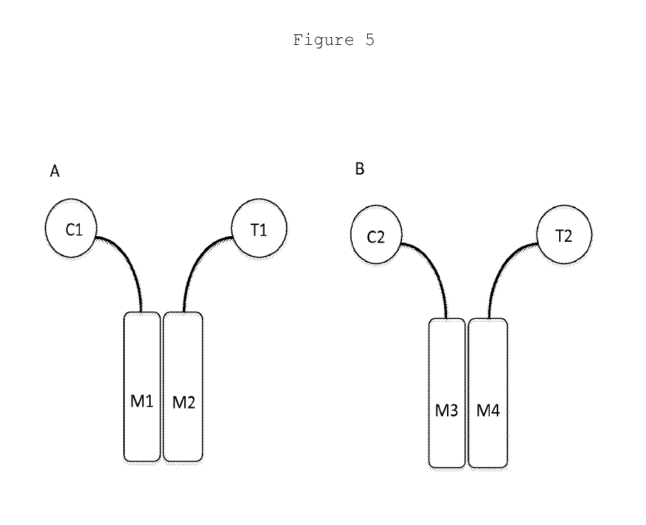

The term “multimerizing domain” refers to a region of a polypeptide that associates with other polypeptides to form complexes. In one aspect, the multimerizing components M1, M2, M3, and M4 may be the same or different. See, e.g., FIG. 5. In another aspect, the multimerizing component is selected from a leucine zipper, a zinc finger, an immunoglobulin light chain constant domain, and an Fc domain. Preferably, at least one of M1, M2, M3, or M4 comprises an Fc domain of an immunoglobulin.

The immunoglobulin light chain constant domain may be a kappa chain or a lambda chain. The kappa chain or lambda chain may be a human kappa chain or lambda chain. The multimerizing component may be an Fc of an IgG. The Fc may be from an IgG of isotype IgG1, IgG2, IgG3, or IgG4. The multimerizing component may comprise a sequence selected from a human IgG1, a human IgG2, a human IgG3, a human IgG4, and a combination thereof. Further, the multimerizing component may contain a C_{H}2 and a C_{H}3 of a human IgG selected from IgG1, IgG2, IgG3, and IgG4.

Preferably, at least one of M1, M2, M3, and M4 is a polypeptide comprising an immunoglobulin C_{H}2 domain or an immunoglobulin C_{H}3 domain. The multimerizing component may contain a C_{H}2 and a C_{H}3 of a human IgG1, IgG2, IgG3, or IgG4, and may be modified as described herein. The immunoglobulin heavy chain constant domain or multimerizing fragment thereof may be human. M1, M2, M3, and M4 each independently may include an immunoglobulin heavy chain constant domain selected from C_{H}2, C_{H}3, and combinations thereof. M1, M2, M3, and M4 each independently may comprise a human C_{H}2 and C_{H}3, arranged, e.g., as found in a human Fc, e.g., in a human IgG1, IgG2, IgG3, or IgG4 Fc. M1, M2, M3, and M4 may comprise immunoglobulin constant domains, or multimerizing portions thereof, that are differentially modified, i.e., modifications present in M1 are not present in M2, M3, or M4, modifications present in M2 are not present in M1, M3, or M4, modifications present in M3 are not present in M1, M2, or M4, or modifications present in M4 are not present in M1, M2, or M3. Unless otherwise specified, modifications that are disclosed herein in connection with M1 may be used with M2, M3, or M4, and so on. That is, e.g., the modifications mentioned throughout for M1 may be used on M2, M3, or M4, and the modifications mentioned throughout for M2 may be used on M1, M3 and M4. At least one of M1, M2, M3, and M4 may comprise an immunoglobulin heavy chain constant domain that comprises a C_{H}3 region of a human IgG selected from IgG1, IgG2, IgG4, and a combination thereof, wherein the C_{H}3 region comprises a modification that reduces or eliminates binding of the second C_{H}3 domain to protein A.

In the present invention, a non-limiting example of the structure of a multi-specific antigen-binding polypeptide includes a first and a second polypeptide, the first polypeptide comprising, from N-terminal to C-terminal, a cell-specific antigen binding domain, followed by a constant region that comprises a first C_{H}3 region of a human IgG selected from IgG1, IgG2, IgG4, and combinations thereof; and, a second polypeptide comprising, from N-terminal to C-terminal, a target antigen binding domain, followed by a constant region that comprises a second C_{H}3 region of a human IgG selected from IgG1, IgG2, IgG4, and combinations thereof, wherein the second C_{H}3 region comprises a modification that reduces or eliminates binding of the second C_{H}3 domain to protein A.

The second C_{H}3 region may comprise an H95R modification (by IMGT exon numbering; H435R by EU numbering). In another example, the second C_{H}3 region further comprises a Y96F modification (IMGT; Y436F by EU).

In the present invention, the second C_{H}3 region may be from a modified human IgG1, and further comprise a modification selected from the group consisting of D16E, L18M, N44S, K52N, V57M, and V82I (IMGT; D356E, L358M, N384S, K392N, V397M, and V422I by EU).

In another example, the second C_{H}3 region may be from a modified human IgG2, and further comprise a modification selected from the group consisting of N44S, K52N, and V82I (IMGT; N384S, K392N, and V422I by EU).

In a further example, the second C_{H}3 region may be from a modified human IgG4, and further comprise a modification selected from the group consisting of QI5R, N44S, K52N, V57M, R69K, E79Q, and V82I (IMGT; Q355R, N384S, K392N, V397M, R409K, E419Q, and V422I by EU).

In another aspect of this embodiment, at least one of C1, C2, T1 or T2 comprises an epitope-binding domain selected from the group consisting of: (i) a Fab; (ii) an scFv; (iii) a diabody (dAb); (iv) a V_{H}/C_{H}1; (v) a V_{L}/C_{L}; and (vi) a domain antibody. Preferably, the epitope-binding domain is (i) a Fab or (ii) an scFv.

In a further aspect of this embodiment, at least one of the first and second multi-specific antigen-binding polypeptides is a bispecific antibody. As used herein, a “bispecific antibody” means a polypeptide that is capable of selectively binding two epitopes. Bispecific antibodies may comprise two different heavy chains, with each heavy chain specifically binding a different epitope-either on two different molecules (e.g., antigens) or on the same molecule (e.g., on the same antigen). The affinity of the bispecific antibody for one epitope is preferably at least one to two or three or four orders of magnitude lower than the affinity of bispecific antibody for the second epitope. The epitopes recognized by the bispecific antibody can be on the same or a different target (e.g., on the same or a different protein). Bispecific antibodies can be made, for example, by combining heavy chains that recognize different epitopes of the same antigen. For example, nucleic acid sequences encoding heavy chain variable sequences that recognize different epitopes of the same antigen can be fused to nucleic acid sequences encoding different heavy chain constant regions, and such sequences can be expressed in a cell that expresses an immunoglobulin light chain. A typical bispecific antibody has two heavy chains each having three heavy chain CDRs, followed by (N-terminal to C-terminal) a C_{H}1 domain, a hinge, a C_{H}2 domain, and a C_{H}3 domain, and an immunoglobulin light chain that either does not confer antigen-binding specificity but that can associate with each heavy chain, or that can associate with each heavy chain and that can bind one or more of the epitopes bound by the heavy chain antigen-binding regions, or that can associate with each heavy chain and enable binding of one or both of the heavy chains to one or both epitopes.

As noted above, one of the antigen binding domains of at least one bispecific antibody has at least a two fold lower affinity, such as three, four, five, six, seven, eight, nine, ten, twenty, thirty, forty, fifty, sixty, seventy, eighty, ninety, one hundred, two hundred, three hundred, four hundred, five hundred, six hundred, seven hundred, eight hundred, nine hundred, one thousand, five thousand, ten thousand, fifty thousand, one hundred thousand, five hundred thousand, or one million fold lower affinity, for its target relative to the other antigen binding domain in the same bispecific antibody.

In the context of the present invention, “affinity” is a measure of the strength of interaction between an antigen-binding domain and an antigen of the present invention. In the present invention, an antibody may be characterized by having specific binding activity (Kₐ) for an antigen of at least about 10⁵ mol⁻¹, 10⁶ mol⁻¹ or greater, preferably 10⁷ mol⁻¹ or greater, more preferably 10⁸ mol⁻¹ or greater, and most preferably 10⁹ mol⁻¹ or greater. The binding affinity of an antibody can be readily determined by one of ordinary skill in the art, for example, by Scatchard analysis (Scatchard, Ann. N.Y. Acad. Sci. 51: 660-72, 1949).

In another aspect of this embodiment, the cell surface density of the cell-specific antigen is lower, such as two, three, four, five, six, seven, eight, nine, ten, twenty, thirty, forty, fifty, sixty, seventy, eighty, ninety, one hundred, two hundred, three hundred, four hundred, five hundred, six hundred, seven hundred, eight hundred, nine hundred, one thousand, five thousand, ten thousand, fifty thousand, one hundred thousand, five hundred thousand, or one million fold lower, than the cell surface density of the target antigen. As used herein, cell surface “density” means the number of molecules, e.g., cell specific antigens, per unit area of the cell surface. Methods for measuring cell surface density are known in the art.

In a further aspect of this embodiment, the first and second multi-specific antigen-binding polypeptides are present in excess, such as three, four, five, six, seven, eight, nine, ten, twenty, thirty, forty, fifty, sixty, seventy, eighty, ninety, one hundred, two hundred, three hundred, four hundred, five hundred, six hundred, seven hundred, eight hundred, nine hundred, one thousand, five thousand, ten thousand, fifty thousand, one hundred thousand, five hundred thousand, or one million fold in excess, relative to the target antigen.

In another aspect of this embodiment, the target antigen is a receptor and modulation is activation of the receptor. In one preferred embodiment, activation of the receptor is ligand dependent. In another preferred embodiment, activation of the receptor is ligand independent. In a further aspect of this embodiment, the target antigen is a receptor and modulation is inhibition of the receptor.

Representative, non-limiting examples of receptors according to the present invention include FcER1a, TrkB, FGFR1c, LIFRb, IL4R, IL2Rgamma, IFNAR1, and IFNAR2.

In another aspect of this embodiment, the cell-specific antigen and the target antigen form a complex on the surface of the cell. In the present invention, a “complex” refers to a group of two or more associated polypeptides, or polypeptides and nucleic acid molecules, or polypeptides and other biological or chemical entities. One of skill in the art will appreciate that such complexes can vary regarding their stability, from very transient to virtually permanent.

In an additional aspect of this embodiment, at least one of C1 and C2 comprises a polypeptide, nucleic acid, or other biological or chemical entity that interacts with the cell-specific antigen, including, but not limited to, a ligand or portion of a ligand of the cell-specific antigen or a polypeptide or portion of a polypeptide that complexes with the cell-specific antigen on the cell surface.

In another aspect of this embodiment, at least one of T1 and T2 comprises a polypeptide, nucleic acid, or other biological or chemical entity that interacts with the target antigen, including, but not limited to, a ligand or portion of a ligand of the target antigen or a polypeptide or portion of a polypeptide that complexes with the target antigen on the cell surface. For example, T1 and/or T2 could be a portion of a ligand and the target antigen could be a receptor for that ligand.

Another embodiment of the present invention is a pharmaceutical composition for cell-specific modulation of a target antigen in a subject in need thereof. The pharmaceutical composition comprises a pharmaceutically acceptable diluent or carrier and an effective amount of:

(a) a first multi-specific antigen-binding polypeptide comprising:
(i) a cell-specific antigen binding domain (C1) and
(ii) a target antigen binding domain (T1); and

(b) a second multi-specific antigen-binding polypeptide comprising:
(i) a cell-specific antigen binding domain (C2) and
(ii) a target antigen binding domain (T2),
   wherein C1 and C2 interact with the same cell-specific antigen, and the cell-specific antigen and the target antigen are on the same target cell.

As used herein, a “subject” is a mammal, preferably, a human. In addition to humans, categories of mammals within the scope of the present invention include, for example, agricultural animals, domestic animals, laboratory animals, etc. Some examples of agricultural animals include cows, pigs, horses, goats, etc. Some examples of domestic animals include dogs, cats, etc. Some examples of laboratory animals include rats, mice, rabbits, guinea pigs, etc.

In this embodiment, the pharmaceutical compositions may be in a unit dosage form comprising both multi-specific antigen-binding polypeptides. In another aspect of this embodiment, the first multi-specific antigen-binding polypeptide is in a first unit dosage form and the second multi-specific antigen-binding polypeptide is in a second unit dosage form, separate from the first.

The first and second multi-specific antigen-binding polypeptides may be co-administered to the subject, either simultaneously or at different times, as deemed most appropriate by a physician. If the first and second multi-specific antigen-binding polypeptides are administered at different times, for example, by serial administration, the first multi-specific antigen-binding polypeptide may be administered to the subject before the second multi-specific antigen-binding polypeptide. Alternatively, the second multi-specific antigen-binding polypeptide may be administered to the subject before the first multi-specific antigen-binding polypeptide.

In this embodiment, suitable and preferred structural options for the first and second multi-specific binding polypeptides are as disclosed above.

For example, the first multi-specific antigen-binding polypeptide may further comprise:
(iii) a first multimerizing domain (M1) and optionally a second multimerizing domain (M2);
   and the second multi-specific antigen-binding polypeptide further comprises:
(iv) a first multimerizing domain (M3) and optionally a second multimerizing domain (M4).

The structure and arrangement of M1, M2, M3, and/or M4 according to the present invention are as disclosed above.

Furthermore, at least one of the first and second multi-specific antigen-binding polypeptides may be a bispecific antibody. Relative affinities of one of the antigen binding domains of the bispecific antibody for its target relative to the other antigen binding domain in the same bispecific antibody is as disclosed above.

The pharmaceutical composition, when administered to a subject, may target a receptor and modulate its activity, i.e., by activating or inhibiting the receptor, which may be ligand dependent or independent, as disclosed above. In addition, administration of the pharmaceutical composition to the subject may cause a complex to form on the surface of the target cell.

A further embodiment of the present invention is a kit for cell-specific modulation of a target antigen. The kit comprises, packaged together with instructions for their use:

(a) a first multi-specific antigen-binding polypeptide comprising:
(i) a cell-specific antigen binding domain (C1) and
(ii) a target antigen binding domain (T1); and

(b) a second multi-specific antigen-binding polypeptide comprising:
(i) a cell-specific antigen binding domain (C2) and
(ii) a target antigen binding domain (T2),
   wherein C1 and C2 interact with the same cell-specific antigen, and the cell-specific antigen and the target antigen are on the same target cell.

The kits may also include suitable storage containers, e.g., ampules, vials, tubes, etc., for each multi-specific antigen-binding polypeptide of the present invention (which may e.g., may be in the form of pharmaceutical compositions) and other reagents, e.g., buffers, balanced salt solutions, etc., for use in administering the first and second multi-specific antigen-binding polypeptides to subjects. The multi-specific antigen-binding polypeptides of the invention and other reagents may be present in the kits in any convenient form, such as, e.g., in a solution or in a powder form. The kits may further include a packaging container, optionally having one or more partitions for housing the first and second multi-specific antigen-binding polypeptides or pharmaceutical compositions of the present invention and other optional reagents.

In this embodiment, suitable and preferred structural options for the first and second multispecific binding polypeptides and pharmaceutical compositions containing the same are as disclosed above.

For example, the first multi-specific antigen-binding polypeptide may further comprise:
(iii) a first multimerizing domain (M1) and optionally a second multimerizing domain (M2);
   and the second multi-specific antigen-binding polypeptide further comprises:
(iv) a first multimerizing domain (M3) and optionally a second multimerizing domain (M4).

Suitable and preferred M1, M2, M3, and/or M4 according to the present invention are as disclosed above.

Furthermore, at least one of the first and second multi-specific antigen-binding polypeptides in the kit may be a bispecific antibody. Relative affinities of one of the antigen binding domains of the bispecific antibody for its target relative to the other antigen binding domain in the same bispecific antibody is as disclosed above.

The first and second multi-specific antigen-binding polypeptides of the kit, including pharmaceutical compositions thereof, may target a receptor and modulate its activity, i.e., by activating or inhibiting the receptor, which may be ligand dependent or independent, as disclosed above. In addition, administration of the first and second multi-specific binding polypeptides of the kit, including pharmaceutical compositions thereof, to a subject, preferably a human in need thereof, may cause a complex to form on the surface of the target cell.

An additional embodiment of the present invention is a method for cell-specific modulation of a target antigen. The method comprises contacting a target cell having the target antigen on the surface of the target cell with:

(a) a first multi-specific antigen-binding polypeptide comprising:
(i) a cell-specific antigen binding domain (C1) and
(ii) a target antigen binding domain (T1);

(b) a second multi-specific antigen-binding polypeptide comprising:
(i) a cell-specific antigen binding domain (C2) and
(ii) a target antigen binding domain (T2); and

(c) an antigen-binding polypeptide comprising:
(i) a first cell-specific antigen binding domain (C3) and
(ii) a second cell-specific antigen binding domain (C4),
   wherein the cell-specific antigens and the target antigen are on the same target cell, C1 and C3 interact with a first cell-specific antigen, and C2 and C4 interact with a second cell-specific antigen.

In one aspect of this embodiment, the first cell-specific antigen and the second cell-specific antigen are a monomers on the cell surface.

In another aspect of this embodiment, the target antigen is composed of at least two copies of the same polypeptide subunit on the cell surface. Preferably, the target antigen is a homodimer on the cell surface.

In a further aspect of this embodiment, the first multi-specific antigen-binding polypeptide further comprises
(i) a first multimerizing domain (M1) and optionally a second multimerizing domain (M2);
the second multi-specific antigen-binding polypeptide further comprises
(ii) a first multimerizing domain (M3) and optionally a second multimerizing domain (M4).
and the antigen-binding polypeptide further comprises
(iii) a first multimerizing domain (M5) and optionally a second multimerizing domain (M6).

The structure and arrangement of the multimerizing domains (M1-M6) are as described above with respect to M1-4. Preferably, however, at least one of M1, M2, M3, M4, M5, and M6 is a polypeptide comprising an immunoglobulin C_{H}2 domain or an immunoglobulin C_{H}3 domain. In another preferred embodiment, at least one of M1, M2, M3, M4, M5, or M6 comprises an Fc domain of an immunoglobulin.

C1-C4 and T1-T2 are as described above. For example, at least one of C1, C2, C3, C4, T1 or T2 may comprise an epitope-binding domain selected from the group consisting of: (i) a Fab; (ii) an scFv; (iii) a dAb; (iv) a V_{H}/CH1; (v) a V_{L}/C_{L}; and (vi) a domain antibody. Preferably, the epitope-binding domain is a Fab or an scFv.

Furthermore, at least one of the first and second multi-specific antigen-binding polypeptides may be a bispecific antibody. Preferably, one of the antigen binding domains of at least one bispecific antibody has at least a two fold lower affinity for its target relative to the other antigen binding domain in the same bispecific antibody.

In a further aspect of this embodiment, the cell surface density of the first and/or the second cell-specific antigen is lower than the cell surface density of the target antigen.

In another aspect of this embodiment, the first and second multi-specific antigen-binding polypeptides are present in excess relative to the target antigen.

In a further aspect of this embodiment, the target antigen is a receptor and modulation is activation of the receptor. In one preferred embodiment, activation of the receptor is ligand dependent. In another preferred embodiment, activation of the receptor is ligand independent.

In another aspect of this embodiment, the target antigen is a receptor and modulation is inhibition of the receptor.

In an additional aspect of this embodiment, at least one of the cell-specific antigens and the target antigen form a complex on the surface of the cell.

In a further aspect of this embodiment, at least one of C1, C2, C3 and C4 comprises a polypeptide, nucleic acid, or other biological or chemical entity that interacts with the cell-specific antigen, including, but not limited to, a ligand or portion of a ligand of the cell-specific antigen or a polypeptide or portion of a polypeptide that complexes with the cell-specific antigen on the cell surface.

In an additional aspect of this embodiment, at least one of T1 and T2 comprises a polypeptide, nucleic acid, or other biological or chemical entity that interacts with the target antigen, including, but not limited to, a ligand or portion of a ligand of the target antigen or a polypeptide or portion of a polypeptide that complexes with the target antigen on the cell surface. For example, T1 and/or T2 could be a portion of a ligand and the target antigen could be a receptor for that ligand.

An additional embodiment of the present invention is a pharmaceutical composition for cell-specific modulation of a target antigen in a subject in need thereof. The pharmaceutical composition comprises a pharmaceutically acceptable diluent or carrier and an effective amount of:

(a) a first multi-specific antigen-binding polypeptide comprising:
(i) a cell-specific antigen binding domain (C1) and
(ii) a target antigen binding domain (T1);

(b) a second multi-specific antigen-binding polypeptide comprising:
(i) a cell-specific antigen binding domain (C2) and
(ii) a target antigen binding domain (T2); and

(c) an antigen-binding polypeptide comprising:
(i) a first cell-specific antigen binding domain (C3) and
(ii) a second cell-specific antigen binding domain (C4), wherein the cell-specific antigens and the target antigen are on the same target cell, C1 and C3 interact with a first cell-specific antigen, and C2 and C4 interact with a second cell-specific antigen.

In this embodiment, suitable and preferred subjects are as disclosed herein.

In this embodiment, the pharmaceutical compositions according to the present invention may be in a unit dosage form comprising both multi-specific antigen-binding polypeptides and the antigen-binding polypeptide. In another aspect of this embodiment, the first multi-specific antigen-binding polypeptide is in a first unit dosage form; the second multi-specific antigen-binding polypeptide is in a second unit dosage form, separate from the first; and the antigen-binding polypeptide is in a third unit dosage form, separate from the first and the second.

The first and second multi-specific antigen-binding polypeptides and the antigen-binding polypeptide of the pharmaceutical composition may be co-administered to the subject, either simultaneously or at different times, as deemed most appropriate by a physician. If the first and second multi-specific antigen-binding polypeptides and the antigen-binding polypeptide are administered at different times, for example, by serial administration, the first and second multi-specific antigen-binding polypeptides may be administered to the subject in any order.

Suitable and preferred structural options for the first and second multi-specific binding polypeptides are as disclosed above.

For example, the first multi-specific antigen-binding polypeptide may further comprise:
(i) a first multimerizing domain (M1) and optionally a second multimerizing domain (M2);

the second multi-specific antigen-binding polypeptide further comprises
(ii) a first multimerizing domain (M3) and optionally a second multimerizing domain (M4).

and the antigen-binding polypeptide further comprises
(iii) a first multimerizing domain (M5) and optionally a second multimerizing domain (M6).

In one preferred embodiment, at least one of M1, M2, M3, M4, M5, and M6 is a polypeptide comprising an immunoglobulin C_{H}2 domain or an immunoglobulin C_{H}3 domain.

In another preferred embodiment, at least one of M1, M2, M3, M4, M5, or M6 comprises an Fc domain of an immunoglobulin.

In a further aspect of this embodiment, at least one of C1, C2, C3, C4, T1 or T2 comprises an epitope-binding domain selected from the group consisting of: (i) a Fab; (ii) an scFv; (iii) a dAb; (iv) a V_{H}/CH1; (v) a V_{L}/C_{L}; and (vi) a domain antibody. Preferably, the epitope-binding domain is a Fab or an scFv.

In another aspect of this embodiment, at least one of the first and second multi-specific antigen-binding polypeptides and/or the antigen-binding polypeptides in the pharmaceutical composition may be a bispecific antibody. Preferably, one of the antigen binding domains of at least one bispecific antibody of the pharmaceutical composition has at least a two fold lower affinity for its target relative to the other antigen binding domain in the same bispecific antibody.

In a further aspect of this embodiment, the cell surface density of at least one cell-specific antigen is lower than of the cell surface density of the target antigen.

In another aspect of this embodiment, the pharmaceutical composition provides the first and second multi-specific antigen-binding polypeptides in excess relative to the target antigen.

In a further aspect of this embodiment, the target antigen is a receptor and the pharmaceutical composition, when administered to a subject, modulates the receptor, e.g., by activating or inhibiting the receptor. In the present invention, the receptor may be ligand dependent or independent.

In an additional aspect of this embodiment, when the pharmaceutical composition is administered to a subject, at least one of the cell-specific antigens and the target antigen form a complex on the surface of the cell.

In a further aspect of this embodiment, at least one of C1, C2, C3 and C4 comprises a polypeptide, nucleic acid, or other biological or chemical entity that interacts with the cell-specific antigen, including, but not limited to, a ligand or portion of a ligand of the cell-specific antigen or a polypeptide or portion of a polypeptide that complexes with the cell-specific antigen on the cell surface.

In an additional aspect of this embodiment, at least one of T1 and T2 comprises a polypeptide, nucleic acid, or other biological or chemical entity that interacts with the target antigen, including, but not limited to, a ligand or portion of a ligand of the target antigen or a polypeptide or portion of a polypeptide that complexes with the target antigen on the cell surface. For example, T1 and/or T2 could be a portion of a ligand and the target antigen could be a receptor for that ligand. An additional embodiment of the present invention is a kit for cell-specific modulation of a target antigen. The kit comprises, packaged together with instructions for their use:

(a) a first multi-specific antigen-binding polypeptide comprising:
(i) a cell-specific antigen binding domain (C1) and
(ii) a target antigen binding domain (T1);

(b) a second multi-specific antigen-binding polypeptide comprising:
(i) a cell-specific antigen binding domain (C2) and
(ii) a target antigen binding domain (T2); and

(c) an antigen-binding polypeptide comprising:
(i) a first cell-specific antigen binding domain (C3) and
(ii) a second cell-specific antigen binding domain (C4), wherein C1 and C3 interact with a first cell-specific antigen, and C2 and C4 interact with a second cell-specific antigen.

In this embodiment, suitable and preferred subjects, first and second multi-specific antigen-binding polypeptides and antigen-binding polypeptides are as disclosed herein. In addition, the first and second multi-specific antigen-binding polypeptides and antigen-binding polypeptides may be in the form of a pharmaceutical composition as described above.

In one aspect of this embodiment, the first and second cell-specific antigens are monomers on the cell surface. In another aspect of this embodiment, the target antigen is composed of at least two copies of the same polypeptide subunit on the cell surface. Preferably, the target antigen is a homodimer on the cell surface.

In a further aspect of this embodiment, the first multi-specific antigen-binding polypeptide further comprises:
(iii) a first multimerizing domain (M1) and optionally a second multimerizing domain (M2);

the second multi-specific antigen-binding polypeptide further comprises
(iii) a first multimerizing domain (M3) and optionally a second multimerizing domain (M4).

and the antigen-binding polypeptide further comprises
(iii) a first multimerizing domain (M5) and optionally a second multimerizing domain (M6).

The structure and arrangement of M1-M6 are as described above. For example, at least one of M1, M2, M3, M4, M5, and M6 may be a polypeptide comprising an immunoglobulin C_{H}2 domain or an immunoglobulin C_{H}3 domain. In another preferred embodiment, at least one of M1, M2, M3, M4, M5, or M6 may comprise an Fc domain of an immunoglobulin.

The structure and arrangement of C1, C2, C3, C4, T1 and T2 are as described above. For example, at least one of C1, C2, T1 or T2 may comprise an epitope-binding domain selected from the group consisting of: (i) a Fab; (ii) an scFv; (iii) a dAb; (iv) a V_{H}/CH1; (v) a V_{L}/C_{L}; and (vi) a domain antibody. Preferably, the epitope-binding domain is a Fab or an scFv.

In another aspect of this embodiment, at least one of the first and second multi-specific antigen-binding polypeptides in the kit is a bispecific antibody. The affinity of the antigen binding domains of the bispecific antibodies is as disclosed above. For example, one of the antigen binding domains of at least one bispecific antibody has at least a two fold lower affinity for its target relative to the other antigen binding domain in the same bispecific antibody.

In a further aspect of this embodiment, the cell surface density of the first and/or the second cell-specific antigen is lower than the cell surface density of the target antigen.

In another aspect of this embodiment, the pharmaceutical composition provides the first and second multi-specific antigen-binding polypeptides in excess relative to the target antigen.

In a further aspect of this embodiment, the target antigen is a receptor and the pharmaceutical composition, when administered to a subject, modulates the receptor, e.g., by activating or inhibiting the receptor. In the present invention, the receptor may be ligand dependent or independent.

In an additional aspect of this embodiment, when the active agents of the kit are administered to a subject, at least one of the cell-specific antigens and the target antigen form a complex on the surface of the cell.

In a further aspect of this embodiment, at least one of C1, C2, C3, and C4 comprises a ligand or portion of a receptor that specifically binds the cell-specific antigen.

In an additional aspect of this embodiment, at least one of T1 and T2 comprises a ligand or portion of a receptor that specifically binds the target antigen.

In the present invention, an “effective amount” or a “therapeutically effective amount” of any of the antigen-binding polypeptides of the invention, whether or not they are multi-specific, including pharmaceutical compositions containing same (hereinafter referred to as “polypeptides of the invention” or simply “polypeptides” unless the context suggests otherwise), is an amount of such a polypeptide that is sufficient to effect beneficial or desired results as described herein when administered to a subject or provided to a cell. Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of mammal, e.g., human patient, and like factors well known in the arts of medicine and veterinary medicine. In general, a suitable dose of a polypeptide or a pharmaceutical composition containing the same according to the invention will be that amount of a polypeptide or a pharmaceutical composition, which is the lowest dose effective to produce the desired effect. The effective dose of a polypeptide or a pharmaceutical composition containing the same of the present invention may be administered as two, three, four, five, six or more sub-doses, administered separately at appropriate intervals throughout the day.

A suitable, non-limiting example of a dosage of a polypeptide or a pharmaceutical composition containing the same disclosed herein may vary depending upon the age and the size of a subject to be administered, target disease, the purpose of the treatment, conditions, route of administration, and the like. In an adult patient, it is advantageous to intravenously or subcutaneously administer the antibody of the present invention at a single dose of about 0.01 to about 20 mg/kg body weight, more preferably about 0.02 to about 7, about 0.03 to about 5, or about 0.05 to about 3 mg/kg body weight. Depending on the severity of the condition, the frequency and the duration of the treatment can be adjusted. In certain embodiments, polypeptide or a pharmaceutical composition containing the same disclosed herein can be administered as an initial dose of at least about 0.1 mg to about 800 mg, about 1 to about 500 mg, about 5 to about 300 mg, or about 10 to about 200 mg, to about 100 mg, or to about 50 mg. In certain embodiments, the initial dose may be followed by administration of a second or a plurality of subsequent doses of the polypeptide or a pharmaceutical composition containing the same in an amount that can be approximately the same or less than that of the initial dose, wherein the subsequent doses are separated by at least 1 day to 3 days; at least one week, at least 2 weeks; at least 3 weeks; at least 4 weeks; at least 5 weeks; at least 6 weeks; at least 7 weeks; at least 8 weeks; at least 9 weeks; at least 10 weeks; at least 12 weeks; or at least 14 weeks.

The polypeptides or pharmaceutical compositions containing same of the present invention may be administered in any desired and effective manner: for oral ingestion, or as an ointment or drop for local administration to the eyes, or for parenteral or other administration in any appropriate manner such as intraperitoneal, subcutaneous, topical, intradermal, inhalation, intrapulmonary, rectal, vaginal, sublingual, intramuscular, intravenous, intraarterial, intrathecal, or intralymphatic. Further, the polypeptides or pharmaceutical compositions containing same of the present invention may be administered in conjunction with other treatments. The polypeptides or the pharmaceutical compositions of the present invention may be encapsulated or otherwise protected against gastric or other secretions, if desired.

The pharmaceutical compositions of the invention comprise one or more active ingredients, e.g. polypeptides, in admixture with one or more pharmaceutically-acceptable diluents or carriers and, optionally, one or more other compounds, drugs, ingredients and/or materials. Regardless of the route of administration selected, the polypeptides of the present invention are formulated into pharmaceutically-acceptable dosage forms by conventional methods known to those of skill in the art. See, e.g., Remington, The Science and Practice of Pharmacy (21^{st} Edition, Lippincott Williams and Wilkins, Philadelphia, Pa.).

Pharmaceutically acceptable diluents or carriers are well known in the art (see, e.g., Remington, The Science and Practice of Pharmacy (21^{st} Edition, Lippincott Williams and Wilkins, Philadelphia, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and tryglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, etc. Each pharmaceutically acceptable diluent or carrier used in a pharmaceutical composition of the invention must be “acceptable” in the sense of being compatible with the other ingredients of the formulation and not injurious to the subject. Diluents or carriers suitable for a selected dosage form and intended route of administration are well known in the art, and acceptable diluents or carriers for a chosen dosage form and method of administration can be determined using ordinary skill in the art.

The pharmaceutical compositions of the invention may, optionally, contain additional ingredients and/or materials commonly used in pharmaceutical compositions. These ingredients and materials are well known in the art and include (1) fillers or extenders, such as starches, lactose, sucrose, glucose, mannitol, and silicic acid; (2) binders, such as carboxymethylcellulose, alginates, gelatin, polyvinyl pyrrolidone, hydroxypropylmethyl cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethyl cellulose and sodium carbonate; (5) solution retarding agents, such as paraffin; (6) absorption accelerators, such as quaternary ammonium compounds; (7) wetting agents, such as cetyl alcohol and glycerol monostearate; (8) absorbents, such as kaolin and bentonite clay; (9) lubricants, such as talc, calcium stearate, magnesium stearate, solid polyethylene glycols, and sodium lauryl sulfate; (10) suspending agents, such as ethoxylated isostearyl alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethyl cellulose, other polymer matrices, biodegradable polymers, liposomes, microspheres, aluminum monostearate, gelatin, and waxes; (18) opacifying agents; (19) adjuvants; (20) wetting agents; (21) emulsifying and suspending agents; (22), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; and (28) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

The pharmaceutical compositions of the present invention suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations may be prepared by methods known in the art, e.g., by means of conventional pan-coating, mixing, granulation or lyophilization processes.

Solid dosage forms for oral administration (capsules, tablets, pills, dragees, powders, granules and the like) may be prepared, e.g., by mixing the active ingredient(s) with one or more pharmaceutically-acceptable diluents or carriers and, optionally, one or more fillers, extenders, binders, humectants, disintegrating agents, solution retarding agents, absorption accelerators, wetting agents, absorbents, lubricants, and/or coloring agents. Solid compositions of a similar type may be employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein. They may be sterilized by, for example, filtration through a bacteria-retaining filter. These compositions may also optionally contain opacifying agents and may be of a composition such that they release the active ingredient only, or preferentially, in a certain portion of the gastrointestinal tract, optionally, in a delayed manner. The active ingredient can also be in microencapsulated form.

Liquid dosage forms for oral administration include pharmaceutically-acceptable emulsions, microemulsions, solutions, suspensions, syrups and elixirs. The liquid dosage forms may contain suitable inert diluents commonly used in the art. Besides inert diluents, the oral compositions may also include adjuvants, such as wetting agents, emulsifying and suspending agents, sweetening, flavoring, coloring, perfuming and preservative agents. Suspensions may contain suspending agents.

The pharmaceutical compositions of the present invention for rectal or vaginal administration may be presented as a suppository, which may be prepared by mixing one or more active ingredient(s) with one or more suitable nonirritating diluents or carriers which are solid at room temperature, but liquid at body temperature and, therefore, will melt in the rectum or vaginal cavity and release the active compound. The pharmaceutical compositions of the present invention which are suitable for vaginal administration also include pessaries, tampons, creams, gels, pastes, foams or spray formulations containing such pharmaceutically-acceptable diluents or carriers as are known in the art to be appropriate.

Dosage forms for the topical or transdermal administration include powders, sprays, ointments, pastes, creams, lotions, gels, solutions, patches, drops and inhalants. The active agent(s)/compound(s) may be mixed under sterile conditions with a suitable pharmaceutically-acceptable diluent or carrier. The ointments, pastes, creams and gels may contain excipients. Powders and sprays may contain excipients and propellants.

Various other delivery systems are known and can be used to administer the pharmaceutical composition of the invention, e.g., encapsulation in liposomes, microparticles, microcapsules, recombinant cells capable of expressing the mutant viruses, receptor mediated endocytosis (see, e.g., Wu et al. (1987) J. Biol. Chem. 262:4429-4432). Methods of introduction include, but are not limited to, intradermal, intramuscular, intraperitoneal, intravenous, subcutaneous, intranasal, epidural, and oral routes. The composition may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

The pharmaceutical composition can be also delivered in a vesicle, in particular a liposome (see Langer (1990) Science 249:1527-1533; Treat et al. (1989) in Liposomes in the Therapy of Infectious Disease and Cancer, Lopez Berestein and Fidler (eds.), Liss, New York, pp. 353-365; Lopez-Berestein, ibid., pp. 317-327; see generally ibid.).

In certain situations, the pharmaceutical composition can be delivered in a controlled release system. In one embodiment, a pump may be used (see Langer, supra; Sefton (1987) CRC Crit. Ref Biomed. Eng. 14:201). In another embodiment, polymeric materials can be used; see, Medical Applications of Controlled Release, Langer and Wise (eds.), CRC Pres., Boca Raton, Fla. (1974). In yet another embodiment, a controlled release system can be placed in proximity of the composition's target, thus requiring only a fraction of the systemic dose (see, e.g., Goodson, in Medical Applications of Controlled Release, supra, vol. 2, pp. 115-138, 1984).

The injectable preparations may include dosage forms for intravenous, subcutaneous, intracutaneous and intramuscular injections, drip infusions, etc. These injectable preparations may be prepared by methods publicly known. For example, the injectable preparations may be prepared, e.g., by dissolving, suspending or emulsifying the antibody or its salt described above in a sterile aqueous medium or an oily medium conventionally used for injections. As the aqueous medium for injections, there are, for example, physiological saline, an isotonic solution containing glucose and other auxiliary agents, etc., which may be used in combination with an appropriate solubilizing agent such as an alcohol (e.g., ethanol), a polyalcohol (e.g., propylene glycol, polyethylene glycol), a nonionic surfactant [e.g., polysorbate 80, HCO-50 (polyoxyethylene (50 mol) adduct of hydrogenated castor oil)], etc. As the oily medium, there are employed, e.g., sesame oil, soybean oil, etc., which may be used in combination with a solubilizing agent such as benzyl benzoate, benzyl alcohol, etc. The injection thus prepared is preferably filled in an appropriate ampoule. A pharmaceutical composition of the present invention can be delivered subcutaneously or intravenously with a standard needle and syringe. In addition, with respect to subcutaneous delivery, a pen delivery device readily has applications in delivering a pharmaceutical composition of the present invention. Such a pen delivery device can be reusable or disposable. A reusable pen delivery device generally utilizes a replaceable cartridge that contains a pharmaceutical composition. Once all of the pharmaceutical composition within the cartridge has been administered and the cartridge is empty, the empty cartridge can readily be discarded and replaced with a new cartridge that contains the pharmaceutical composition. The pen delivery device can then be reused. In a disposable pen delivery device, there is no replaceable cartridge. Rather, the disposable pen delivery device comes prefilled with the pharmaceutical composition held in a reservoir within the device. Once the reservoir is emptied of the pharmaceutical composition, the entire device is discarded.

Numerous reusable pen and autoinjector delivery devices have applications in the subcutaneous delivery of a pharmaceutical composition of the present invention. Examples include, but certainly are not limited to AUTOPEN™ (Owen Mumford, Inc., Woodstock, UK), DISETRONIC™ pen (Disetronic Medical Systems, Burghdorf, Switzerland), HUMALOG MIX 75/25™ pen, HUMALOG™ pen, HUMALIN 70/30™ pen (Eli Lilly and Co., Indianapolis, Ind.), NOVOPEN™ I, II and III (Novo Nordisk, Copenhagen, Denmark), NOVOPEN JUNIOR™ (Novo Nordisk, Copenhagen, Denmark), BD™ pen (Becton Dickinson, Franklin Lakes, N.J.), OPTIPENT™, OPTIPEN PRO™, OPTIPEN STARLET™, and OPTICLIK™ (sanofi-aventis, Frankfurt, Germany), to name only a few. Examples of disposable pen delivery devices having applications in subcutaneous delivery of a pharmaceutical composition of the present invention include, but certainly are not limited to the SOLOSTAR™ pen (sanofi-aventis), the FLEXPEN™ (Novo Nordisk), and the KWIKPEN™ (Eli Lilly).

Advantageously, the pharmaceutical compositions for oral or parenteral use described above are prepared into dosage forms in a unit dose suited to fit a dose of the active ingredients. Such dosage forms in a unit dose include, for example, tablets, pills, capsules, injections (ampoules), suppositories, etc. The amount of the aforesaid antibody contained is generally about 0.1 to about 800 mg per dosage form in a unit dose; especially in the form of injection, the aforesaid antibody is contained in about 1 to about 500 mg, in about 5 to 300 mg, in about 8 to 200 mg, and in about 10 to about 100 mg for the other dosage forms.

In some cases, in order to prolong the effect of a drug (e.g., pharmaceutical formulation), it is desirable to slow its absorption from subcutaneous or intramuscular injection. This may be accomplished by the use of a liquid suspension of crystalline or amorphous material having poor water solubility.

The rate of absorption of the active agent/drug then depends upon its rate of dissolution which, in turn, may depend upon crystal size and crystalline form. Alternatively, delayed absorption of a parenterally-administered agent/drug may be accomplished by dissolving or suspending the active agent/drug in an oil vehicle. Injectable depot forms may be made by forming microencapsule matrices of the active ingredient in biodegradable polymers. Depending on the ratio of the active ingredient to polymer, and the nature of the particular polymer employed, the rate of active ingredient release can be controlled. Depot injectable formulations are also prepared by entrapping the drug in liposomes or microemulsions which are compatible with body tissue. The injectable materials can be sterilized for example, by filtration through a bacterial-retaining filter.

The formulations may be presented in unit-dose or multi-dose sealed containers, for example, ampules and vials, and may be stored in a lyophilized condition requiring only the addition of the sterile liquid diluent or carrier, for example water for injection, immediately prior to use. Extemporaneous injection solutions and suspensions may be prepared from sterile powders, granules and tablets of the type described above.

### ADDITIONAL DEFINITIONS

As used herein, terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymers.

As applied to polypeptides, the term “substantial similarity” or “substantially similar” means that two peptide sequences, when optimally aligned, such as by the programs GAP or BESTFIT using default gap weights, share at least 90% sequence identity, even more preferably at least 95%, 98% or 99% sequence identity. Preferably, residue positions, which are not identical, differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a polypeptide. In cases where two or more amino acid sequences differ from each other by conservative substitutions, the percent or degree of similarity may be adjusted upwards to correct for the conservative nature of the substitution. Means for making this adjustment are well known to those of skill in the art. See, e.g., Pearson (1994) Methods Mol. Biol. 24: 307-331, which is herein incorporated by reference. Examples of groups of amino acids that have side chains with similar chemical properties include 1) aliphatic side chains: glycine, alanine, valine, leucine and isoleucine; 2) aliphatic-hydroxyl side chains: serine and threonine; 3) amide-containing side chains: asparagine and glutamine; 4) aromatic side chains: phenylalanine, tyrosine, and tryptophan; 5) basic side chains: lysine, arginine, and histidine; 6) acidic side chains: aspartate and glutamate, and 7) sulfur-containing side chains: cysteine and methionine. Preferred conservative amino acids substitution groups are: valine-leucine-isoleucine, phenylalanine-tyrosine, lysine-arginine, alanine-valine, glutamate-aspartate, and asparagine-glutamine. Alternatively, a conservative replacement is any change having a positive value in the PAM250 log-likelihood matrix disclosed in Gonnet et al. (1992) Science 256: 1443 45, herein incorporated by reference. A “moderately conservative” replacement is any change having a nonnegative value in the PAM250 log-likelihood matrix.

Sequence similarity for polypeptides is typically measured using sequence analysis software. Protein analysis software matches similar sequences using measures of similarity assigned to various substitutions, deletions and other modifications, including conservative amino acid substitutions. For instance, GCG software contains programs such as GAP and BESTFIT which can be used with default parameters to determine sequence homology or sequence identity between closely related polypeptides, such as homologous polypeptides from different species of organisms or between a wild type protein and a mutein thereof. See, e.g., GCG Version 6.1. Polypeptide sequences also can be compared using FASTA with default or recommended parameters; a program in GCG Version 6.1. FASTA (e.g., FASTA2 and FASTA3) provides alignments and percent sequence identity of the regions of the best overlap between the query and search sequences (Pearson (2000) supra). Another preferred algorithm when comparing a sequence of the invention to a database containing a large number of sequences from different organisms is the computer program BLAST, especially BLASTP or TBLASTN, using default parameters. See, e.g., Altschul et al. (1990) J. Mol. Biol. 215: 403-410 and (1997) Nucleic Acids Res. 25:3389-3402, each of which is herein incorporated by reference.

The term “amino acid” means naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, gamma-carboxyglutamate, and O-phosphoserine. An “amino acid analog” means compounds that have the same basic chemical structure as a naturally occurring amino acid, e.g., a carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. An “amino acid mimetic” means a chemical compound that has a structure that is different from the general chemical structure of an amino acid, but that functions similarly to a naturally occurring amino acid.

The term “antibody”, as used herein, is intended to refer to immunoglobulin molecules comprised of four polypeptide chains, two heavy (H) chains and two light (L) chains inter-connected by disulfide bonds (i.e., “full antibody molecules”), as well as multimers thereof (e.g. IgM) or antigen-binding fragments thereof. Each heavy chain is comprised of a heavy chain variable region (“HCVR” or “V_{H}”) and a heavy chain constant region (comprised of domains CH1, CH2 and CH3). Each light chain is comprised of a light chain variable region (“LCVR or “V_{L}”) and a light chain constant region (C_{L}). The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In certain embodiments of the invention, the FRs of the antibody (or antigen binding fragment thereof) may be identical to the human germline sequences, or may be naturally or artificially modified. An amino acid consensus sequence may be defined based on a side-by-side analysis of two or more CDRs.

Substitution of one or more CDR residues or omission of one or more CDRs is also possible. Antibodies have been described in the scientific literature in which one or two CDRs can be dispensed with for binding. Padlan et al. (1995 FASEB J. 9:133-139) analyzed the contact regions between antibodies and their antigens, based on published crystal structures, and concluded that only about one fifth to one third of CDR residues actually contact the antigen. Padlan also found many antibodies in which one or two CDRs had no amino acids in contact with an antigen (see also, Vajdos et al. 2002 J Mol Biol 320:415-428).

CDR residues not contacting antigen can be identified based on previous studies (for example residues H60-H65 in CDRH2 are often not required), from regions of Kabat CDRs lying outside Chothia CDRs, by molecular modeling and/or empirically. If a CDR or residue(s) thereof is omitted, it is usually substituted with an amino acid occupying the corresponding position in another human antibody sequence or a consensus of such sequences. Positions for substitution within CDRs and amino acids to substitute can also be selected empirically. Empirical substitutions can be conservative or non-conservative substitutions.

Full length antibodies can be proteolytically digested down to several discrete, functional antibody fragments, which retain the ability to recognize the antigen. For example, the enzyme papain can be used to cleave a full length immunoglobulin into two Fab fragments and an Fc fragment. Thus, the Fab fragment is typically composed of two variable domains and two constant domains from the heavy and light chains. The Fv region is usually recognized as a component of the Fab region and typically comprises two variable domains, one from each of the heavy (V_{H}) and light (V_{L}) chains. The enzyme pepsin cleaves below the hinge region, so a F(ab′)₂ fragment and a pFc′ fragment is formed. F(ab′)₂ fragments are intact antibodies that have been digested, removing the constant (Fc) region. Two Fab′ fragments can then result from further digestion of F(ab′)₂ fragments. As used herein, “antibody fragments” means a portion of the full length antibody that retains the ability to recognize the antigen, as well as various combinations of such portions. Examples of antibody fragments include, but are not limited to, Fv, Fab, Fab′, Fab′-SH, F(ab′)₂, diabodies, tribodies, scFvs, and single-domain antibodies (sdAbs). Diabodies, tribodies, scFvs, and sdAbs are disclosed in detail below.

The antibodies and, more broadly, the multi-specific antigen-binding polypeptides disclosed herein may comprise one or more amino acid substitutions, insertions and/or deletions in the framework and/or CDR regions of the heavy and light chain variable domains as compared to the corresponding germline sequences. Such mutations can be readily ascertained by comparing the amino acid sequences disclosed herein to germline sequences available from, for example, public antibody sequence databases. The present invention includes antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the V_{H} and/or V_{L} domains are mutated back to the residues found in the original germline sequence from which the antibody was derived. In other embodiments, only certain residues are mutated back to the original germline sequence, e.g., only the mutated residues found within the first 8 amino acids of FR1 or within the last 8 amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present invention may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present invention.

The term “monoclonal antibody”, as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic epitope. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature 256: 495 (1975), and as modified by the somatic hybridization method as set forth above; or may be made by other recombinant DNA methods (see, e.g., U.S. Pat. No. 4,816,567).

The antigen-binding polypeptides of the present invention may be chimeric, humanized or human. For application in man, it is often desirable to reduce immunogenicity of antigen-binding polypeptides, such as, e.g., antibodies, originally derived from other species, like mouse. This can be done, e.g., by construction of chimeric antibodies, or by a process called “humanization”. In this context, a “chimeric antibody” is understood to be an antibody comprising a domain (e.g. a variable domain) derived from one species (e.g. mouse) fused to a domain (e.g. the constant domains) derived from a different species (e.g. human).

As used herein, the term “humanized antibody” refers to forms of antibodies that contain sequences from both non-human (e.g., murine) antibodies as well as human antibodies. Such antibodies are chimeric antibodies which contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the framework (FR) regions are those of a human immunoglobulin sequence. The humanized antibody optionally also will comprise at least a portion of an immunoglobulin constant region (Fc), typically that of a human immunoglobulin (Jones et al., Nature 321:522-525 (1986); Riechmann et al., Nature 332:323-329 (1988); and Presta, Curr. Op. Struct. Biol 2:593-596 (1992)). Humanization can be essentially performed following the method of Winter and co-workers (Jones et al., Nature 321:522-525 (1986); Riechmann et al., Nature 332:323-3′27 (1988); Verhoeyen et al., Science 239:1534-1536 (1988)), by substituting rodent CDRs or CDR sequences for the corresponding sequences of a human antibody.

Furthermore, technologies have been developed for creating antibodies based on sequences derived from the human genome, for example by phage display or using transgenic animals (WO 90/05144; D. Marks, H. R. Hoogenboom, T. P. Bonnert, J. McCafferty, A. D. Griffiths and G. Winter (1991) “By-passing immunization. Human antibodies from V-gene libraries displayed on phage.” J. Mol. Biol., 222, 581-597; Knappik et al., J. Mol. Biol. 296: 57-86, 2000; S. Carmen and L. Jermutus, “Concepts in antibody phage display”. Briefings in Functional Genomics and Proteomics 2002 1(2):189-203; Lonberg N, Huszar D. “Human antibodies from transgenic mice”. Int Rev Immunol. 1995; 13(1):65-93; Bruggemann M, Taussig M J. “Production of human antibody repertoires in transgenic mice”. Curr Opin Biotechnol. 1997 August; 8(4):455-8.). Such antibodies are “human antibodies” in the context of the present invention. Specifically, methods for generating human antibodies in genetically modified mice are known (see e.g., U.S. Pat. No. 6,596,541, Regeneron Pharmaceuticals, VELOCIMMUNE®). The VELOCIMMUNE® technology involves generation of a genetically modified mouse having a genome comprising human heavy and light chain variable regions operably linked to endogenous mouse constant region loci such that the mouse produces an antibody comprising a human variable region and a mouse constant region in response to antigenic stimulation. The DNA encoding the variable regions of the heavy and light chains of the antibodies produced from a VELOCIMMUNE® mouse are fully human. Initially, high affinity chimeric antibodies are isolated having a human variable region and a mouse constant region. The antibodies are characterized and selected for desirable characteristics, including affinity, selectivity, epitope, etc. The mouse constant regions are replaced with a desired human constant region to generate a fully human antibody containing a non-IgM isotype, for example, wild type or modified IgG1, IgG2, IgG3, or IgG4. While the constant region selected may vary according to specific use, high affinity antigen-binding and target specificity characteristics reside in the variable region.

As used herein, “recombinant” antibody means any antibody whose production involves expression of a non-native DNA sequence encoding the desired antibody structure in an organism. In the present invention, recombinant antibodies include tandem scFv (taFv or scFv₂), diabody, dAb₂/VHH₂, knob-into-holes derivates, SEED-IgG, heteroFc-scFv, Fab-scFv, scFv-Jun/Fos, Fab′-Jun/Fos, tribody, DNL-F(ab)₃, scFv₃-CH1/C_{L}, Fab-scFv₂, IgG-scFab, IgG-scFv, scFv-IgG, scFv₂-Fc, F(ab′)₂-scFv₂, scDB-Fc, scDb-CH3, Db-Fc, scFv₂-H/L, DVD-Ig, tandAb, scFv-dhlx-scFv, dAb₂-IgG, dAb-IgG, dAb-Fc-dAb, and combinations thereof.

The present invention also includes fully human antibodies comprising variants of any of the HCVR, LCVR, and/or CDR amino acid sequences disclosed herein having one or more conservative substitutions. For example, the present invention includes antibodies having HCVR, LCVR, and/or CDR amino acid sequences with, e.g., 10 or fewer, 8 or fewer, 6 or fewer, 4 or fewer, etc. conservative amino acid substitutions relative to any of the HCVR, LCVR, and/or CDR amino acid sequences disclosed herein.

The term “human antibody”, as used herein, is intended to include antibodies having variable and constant regions derived from human germline immunoglobulin sequences. The human mAbs of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, as used herein, is not intended to include mAbs in which CDR sequences derived from the germline of another mammalian species (e.g., mouse), have been grafted onto human FR sequences.

Though most naturally occurring antibodies are composed of heavy chains and light chains, camelids (e.g. camels, dromedaries, llamas, and alpacas) and some sharks produce antibodies that consist only of heavy chains. These antibodies bind antigenic epitopes using a single variable domain known as VHH. When produced in Escherichia coli, these molecules are termed single domain antibodies (sdAbs). The simplest application of sdAbs in bispecific antibodies is to link two different sdAbs together to form dAb₂s (VHH₂s). sdAbs can also be applied to IgG-like bispecific antibodies. Examples of this include, but are not limited to, sdAb₂-IgGs, sdAb-IgGs, and sdAb-Fc-dAbs. sdAb₂-IgGs have a similar structure to intact antibodies, but with sdAbs linked to the N-terminal end of the molecule. sdAb-IgGs are intact antibodies specific for one epitope with a single sdAb specific for another epitope linked to the N-termini or C-termini of the heavy chains. Lastly, sdAb-Fc-sdAbs are Fc domains with sdAbs specific for one epitope linked to the N-termini and sdAbs specific for another epitope linked to the C-termini (Chames, P. and Baty, D. In: Bispecific Antibodies. Kontermann R E (ed.), Springer Heidelberg Dordrecht London New York, pp. 101-114 (2011)). Each of the foregoing antibodies is within the scope of the present invention.

Antigen-binding fragments may include antibody fragments, such as a Fab fragment, a F(ab′)2 fragment, a Fv fragment, a dAb fragment, a fragment containing a CDR, or an isolated CDR. In certain embodiments, the term “antigen-binding fragment” refers to a polypeptide fragment of a multi-specific antigen-binding polypeptide. Antigen-binding fragments of an antigen-binding polypeptide, e.g., an antibody, may be derived, e.g., from full antibody molecules using any suitable standard techniques such as proteolytic digestion or recombinant genetic engineering techniques involving the manipulation and expression of DNA encoding antibody variable and (optionally) constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR, which is adjacent to or in frame with one or more framework sequences. In antigen-binding fragments having a VH domain associated with a VL domain, the VH and VL domains may be situated relative to one another in any suitable arrangement. For example, the variable region may be dimeric and contain VH-VH, VH-VL or VL-VL dimers. Alternatively, the antigen-binding fragment of an antibody may contain a monomeric VH or VL domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) VH —CH1; (ii) VH —CH2; (iii) VH —CH3; (iv) VH —CH1-CH2; (v) VH —CH1-CH2-CH3; (vi) VH —CH2-CH3; (vii) VH —CL; (viii) VL —CH1; (ix) VL —CH2; (x) VL —CH3; (xi) VL —CH1-CH2; (xii) VL —CH1-CH2-CH3; (xiii) VL —CH2-CH3; and (xiv) VL —CL. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids, which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric VH or VL domain (e.g., by disulfide bond(s)).

As with full antibody molecules, antigen-binding fragments may be mono-specific or multi-specific (e.g., bi-specific). A multi-specific antigen-binding fragment of an antigen-binding polypeptide, e.g., an antibody, will typically comprise at least two different variable domains, wherein each variable domain is capable of specifically binding to a separate antigen or to a different epitope on the same antigen. Any multi-specific antigen-binding polypeptide format, including the exemplary bi-specific antibody formats disclosed herein, may be adapted for use in the context of an antigen-binding fragment of an antibody of the present invention using routine techniques available in the art.

In specific embodiments, antigen-binding polypeptides or fragments thereof, e.g., an antibody or antibody fragments of the invention, may be conjugated to a moiety such as a ligand or a therapeutic moiety (“immunoconjugate”), such as an antibiotic, a second antibody, or an antibody to another antigen such as a tumor-specific antigen, an autoimmune tissue antigen, a virally-infected cell antigen, a Fc receptor, a T-cell receptor, or a T-cell co-inhibitor, or an immunotoxin, or any other therapeutic moiety useful for treating a disease or condition including cancer, autoimmune disease, or chronic viral infection.

An “isolated antigen-binding polypeptide,” e.g., an “isolated antibody”, as used herein, is intended to refer to an antigen-binding polypeptide, such as an antibody that is substantially free of other antigen-binding polypeptides, e.g., antibodies (Abs), having different antigenic specificities (e.g., an isolated antibody that specifically binds the cell-specific or target antigens of the present invention, or a fragment thereof, is substantially free of Abs that specifically bind antigens other than the cell-specific or target antigens of the present invention).

A “blocking antibody” or a “neutralizing antibody”, as used herein (or an “antibody that neutralizes activity of the cell-specific or target antigens of the present invention” or “antagonist antibody”), is intended to refer to an antibody whose binding to the cell-specific or target antigens of the present invention results in inhibition of at least one biological activity of the cell-specific or target antigens of the present invention.

An “activating antibody” or an “enhancing antibody”, as used herein (or an “agonist antibody”), is intended to refer to an antibody whose binding to the cell-specific or target antigens of the present invention results in increasing or stimulating at least one biological activity of the cell-specific or target antigens of the present invention.

Preferably, the light chains for the multi-specific antigen-binding polypeptides of the invention, such as, e.g., bi-specific or other multi-specific polypeptides are the so-called universal light chains (“ULCs”) as disclosed in, e.g., U.S. patent application Ser. Nos. 13/832,247 and 14/030,424. A common light chain for a plurality of heavy chains has a practical utility. In various embodiments, antigen-binding polypeptides, e.g., antibodies, that are expressed in a mouse that can only express a common light chain will have heavy chains that can associate and express with an identical or substantially identical light chain. This is particularly useful in making multi-specific antigen-binding polypeptides, such as, e.g., bispecific antibodies. The compositions and methods described herein utilize polypeptides that bind more than one epitope with high affinity, e.g., bispecific antibodies. Advantages of this methodology include the ability to select suitably high binding (e.g., affinity matured) heavy chain immunoglobulin chains each of which will associate with a single light chain.

Several techniques for making bispecific antibody fragments from recombinant cell culture have been reported. However, synthesis and expression of bispecific binding polypeptides has been problematic, in part due to issues associated with identifying a suitable light chain that can associate and express with two different heavy chains, and in part due to isolation issues. The ULCs as disclosed in U.S. patent application Ser. Nos. 13/832,247 and 14/030,424 originate from a mouse genetically modified to select, through otherwise natural processes, a suitable light chain that can associate and express with more than one heavy chain, including heavy chains that are somatically mutated (e.g., affinity matured). Human V_{L} and V_{H} sequences from suitable B cells of immunized mice that express affinity matured antibodies having reverse chimeric heavy chains (i.e., human variable and mouse constant) can be identified and cloned in frame in an expression vector with a suitable human constant region gene sequence (e.g., a human IgG1). Two such constructs can be prepared, wherein each construct encodes a human heavy chain variable domain that binds a different epitope. One of the human V_{L}s (e.g., human VK1-39JK5 or human VK3-20JK1), in germline sequence or from a B cell wherein the sequence has been somatically mutated, can be fused in frame to a suitable human constant region gene (e.g., a human K constant gene). These three fully human heavy and light constructs can be placed in a suitable cell for expression. The cell will express two major species: a homodimeric heavy chain with the identical light chain, and a heterodimeric heavy chain with the identical light chain. To allow for a facile separation of these major species, one of the heavy chains is modified to omit a Protein A-binding determinant, resulting in a differential affinity of a homodimeric binding polypeptide from a heterodimeric binding polypeptide. Compositions and methods that address this issue are described in U.S. patent application Ser. No. 12/832,838, filed 25 Jun. 2010, entitled “Readily Isolated Bispecific Antibodies with Native Immunoglobulin Format,” published as US 2010/10331527 A1, hereby incorporated by reference.

One method for making an epitope-binding polypeptide that binds more than one epitope is to immunize a first mouse with an antigen that comprises a first epitope of interest, wherein the mouse comprises an endogenous immunoglobulin light chain variable region locus that does not contain an endogenous mouse V_{L} that is capable of rearranging and forming a light chain, wherein at the endogenous mouse immunoglobulin light chain variable region locus is a single rearranged human V_{L} region operably linked to the mouse endogenous light chain constant region gene, and the rearranged human V_{L} region is selected from a human VK1-39JK5 and a human VK3-20JK1, and the endogenous mouse V_{H} gene segments have been replaced in whole or in part with human V_{H} gene segments, such that immunoglobulin heavy chains made by the mouse are solely or substantially heavy chains that comprise human variable domains and mouse constant domains. When immunized, such a mouse will make a reverse chimeric antibody, comprising only one of two human light chain variable domains (e.g., one of human VK1-39JK5 or human VK3-20JK1). Once a B cell is identified that encodes a V_{H} that binds the epitope of interest, the nucleotide sequence of the V_{H} (and, optionally, the V_{L}) can be retrieved (e.g., by PCR) and cloned into an expression construct in frame with a suitable human immunoglobulin constant domain. This process can be repeated to identify a second V_{H} domain that binds a second epitope, and a second V_{H} gene sequence can be retrieved and cloned into an expression vector in frame to a second suitable immunoglobulin constant domain. The first and the second immunoglobulin constant domains can be of the same or different isotype, and one of the immunoglobulin constant domains (but not the other) can be modified as described herein or in US 2010/0331527 A1, and epitope-binding polypeptide can be expressed in a suitable cell and isolated based on its differential affinity for Protein A as compared to a homodimeric epitope-binding polypeptide, e.g., as described in US 2010/0331527 A1.

Thus, a method for making a bispecific epitope-binding polypeptide comprises identifying a first affinity-matured (e.g., comprising one or more somatic hypermutations) human V_{H} nucleotide sequence (V_{H}1) from a mouse, identifying a second affinity-matured (e.g., comprising one or more somatic hypermutations) human V_{H} nucleotide sequence (V_{H}2) from a mouse as described herein, cloning V_{H}1 in frame with a human heavy chain lacking a Protein A-determinant modification as disclosed, e.g., in US 2010/10331527 A1 for forming heavy chain 1 (HCl), cloning V_{H}2 in frame with a human heavy chain comprising a Protein A-determinant as disclosed, e.g., in US 2010/10331527 A1 to form heavy chain 2 (HC2), introducing an expression vector comprising HCl and the same or a different expression vector comprising HC2 into a cell, wherein the cell also expresses a human immunoglobulin light chain that comprises a human VK1-39/human JK5 or a human VK3-20/human JK1 fused to a human light chain constant domain, allowing the cell to express a bispecific epitope binding polypeptide comprising a V_{H} domain encoded by V_{H}1 and a V_{H} domain encoded by V_{H}2, and isolating the bispecific epitope-binding polypeptide based on its differential ability to bind Protein A as compared with a mono-specific homodimeric epitope-binding polypeptide. In an exemplary embodiment, HCl may be an IgG1, and HC2 may be an IgG1 that comprises the modification H95R (IMGT; H435R by EU) and further comprises the modification Y96F (IMGT; Y436F by EU). In another exemplary embodiment, the V_{H} domain encoded by V_{H}1, the V_{H} domain encoded by V_{H}2, or both, may be somatically mutated.

To express human V_{H} genes that express with a universal human V_{L}, a variety of human variable regions from affinity matured antibodies raised against four different antigens may be expressed with either their cognate light chain, or at least one of a human light chain selected from human VK1-39/JK5, human VK3-20/JK1, or human VpreB/JK5. For antibodies to each of the antigens, somatically mutated high affinity heavy chains from different gene families may be paired successfully with rearranged human germline 39JK5 and VK3-20JKI regions and may be secreted from cells expressing the heavy and light chains. For VK1-39JK5 and VK3-20JKI, V_{H} domains derived from the following human V_{H} gene families may be expressed favorably: 1-2, 1-8, 1-24, 2-5, 3-7, 3-9, 3-11, 3-13, 3-15, 3-20, 3-23, 3-30, 3-33, 3-48, 4-31, 4-39, 4-59, 5-51, and 6-1. Thus, a mouse that is engineered to express a limited repertoire of human V_{L} domains from one or both of VK1-39JK5 and VK3-20JKI will generate a diverse population of somatically mutated human V_{H} domains from a V_{H} locus modified to replace mouse V_{H} gene segments with human V_{H} gene segments.

Mice genetically engineered to express reverse chimeric (human variable, mouse constant) immunoglobulin heavy chains associated with a single rearranged light chain (e.g., a VK1-39/J or a VK3-20/J), when immunized with an antigen of interest, may generate B cells that comprise a diversity of human V_{H} rearrangements and express a diversity of high-affinity antigen-specific antibodies with diverse properties with respect to their ability to block binding of the antigen to its ligand, and with respect to their ability to bind variants of the antigen.

Thus, the mice and methods described herein are useful in making and selecting human immunoglobulin heavy chain variable domains, including somatically mutated human heavy chain variable domains, that result from a diversity of rearrangements, that exhibit a wide variety of affinities (including exhibiting a K_{D} of about a nanomolar or less), a wide variety of specificities (including binding to different epitopes of the same antigen), and that associate and express with the same or substantially the same human immunoglobulin light chain variable region.

To generate fully human multi-specific antigen-binding polypeptide, such as, e.g., bispecific antibodies having a common light chain, as a first step in various embodiments, the first and second nucleic acid sequences that each encode human heavy chain variable domains (and any additional nucleic acid sequences forming the bispecific antibody) are selected from parent monoclonal antibodies having desired characteristics such as, for example, the ability to bind different epitopes, different affinities, etc. Normally, the nucleic acid sequences encoding the human heavy chain variable domains are isolated from immunized mice to allow for fusing with human heavy chain constant regions to be suitable for human administration. Further modifications to the sequence(s) can be made by introducing mutations that add additional functionality to the bispecific antibody, which include, for example, increasing serum half-life (e.g., see U.S. Pat. No. 7,217,797) and/or increasing antibody-dependent cell-mediated cytotoxicity (e.g., see U.S. Pat. No. 6,737,056). Introducing mutations into the constant regions of antibodies is known in the art. Additionally, part of the bispecific antibody can be made recombinantly in cell culture and other part(s) of the molecule can be made by those techniques mentioned above.

Several techniques for producing antigen-binding polypeptides, such as, e.g., antibodies, have been described. For example, in various embodiments chimeric antibodies are produced in mice as described herein. Antibodies can be isolated directly from B cells of an immunized mouse (e.g., see US 2007/0280945 A1) and/or the B cells of the immunized mouse can be used to make hybridomas (Kohler and Milstein, 1975, Nature 256:495-497). DNA encoding the antibodies (human heavy and/or light chains) from mice is readily isolated and sequenced using conventional techniques. Hybridoma and/or B cells derived from mice serve as a preferred source of such DNA. Once isolated, the DNA may be placed into expression vectors, which are then transfected into host cells that do not otherwise produce immunoglobulin polypeptide, to obtain the synthesis of monoclonal antibodies in the recombinant host cells. The DNA also may be modified, for example, by substituting the coding sequence for human heavy and light chain constant domains in place of the murine sequences.

In various embodiments, following isolation of the DNA and selection of the first and second nucleic acid sequences that encode the first and second human heavy chain variable domains having the desired specificities/affinities, and a third nucleic acid sequence that encodes a human light chain domain (a germline rearranged sequence or a light chain sequence isolated from a mouse as described herein), the three nucleic acid sequences encoding the molecules are expressed to form a bispecific antibody using recombinant techniques which are widely available in the art. Often, the expression system of choice will involve a mammalian cell expression vector and host so that the bispecific antibody is appropriately glycosylated (e.g., in the case of bispecific antibodies comprising antibody domains which are glycosylated). However, the molecules can also be produced in the prokaryotic expression systems. Normally, the host cell will be transformed with DNA encoding both the first human heavy chain variable domain, the second human heavy chain variable domain, the human light chain domain on a single vector or independent vectors. However, it is possible to express the first human heavy chain variable domain, second human heavy chain variable domain, and human light chain domain (the bispecific antibody components) in independent expression systems and couple the expressed polypeptides in vitro. In various embodiments, the human light chain domain comprises a germline sequence. In various embodiments, the human light chain domain comprises no more than one, no more than two, no more than three, no more than four, or no more than five somatic hypermutations with the light chain variable sequence of the light chain domain.

In various embodiments, the nucleic acid(s) (e.g., cDNA or genomic DNA) encoding the two heavy chains and single human light chain are inserted into a replicable vector for further cloning (amplification of the DNA) and/or for expression. Many vectors are available, and generally include, but are not limited to, one or more of the following: a signal sequence, an origin of replication, one or more marker genes, an enhancer element, a promoter, and a transcription termination sequence. Each component may be selected individually or based on a host cell choice or other criteria determined experimentally. Several examples of each component are known in the art.

Expression and cloning vectors usually contain a promoter that is recognized by the host organism and is operably linked to the nucleic acid sequences that encode each or all the components of the bispecific antibody. A large number of promoters recognized by a variety of potential host cells are well known. These promoters are operably linked to bispecific antibody-encoding DNA by removing the promoter from the source DNA by restriction enzyme digestion and inserting the isolated promoter sequence into the vector. Expression vectors used in eukaryotic host cells (yeast, fungi, insect, plant, animal, human, or nucleated cells from other multicellular organisms) may also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding the bispecific antibody components. Suitable expression vectors for various embodiments include those that provide for the transient expression in mammalian cells of DNA encoding the bispecific antibody. In general, transient expression involves the use of an expression vector that is able to replicate efficiently in a host cell, such that the host cell accumulates many copies of the expression vector and, in turn, synthesizes high levels of a desired polypeptide encoded by the expression vector. Transient expression systems, comprising a suitable expression vector and a host cell, allow for the convenient positive identification of polypeptides encoded by cloned DNAs, as well as for the rapid screening of bispecific antibodies having desired binding specificities/affinities or the desired gel migration characteristics relative to the parental antibodies having homodimers of the first or second human heavy chain variable domains. In various embodiments, once the DNA encoding the components of the bispecific antibody are assembled into the desired vector(s) as described above, they are introduced into a suitable host cell for expression and recovery. Transfecting host cells can be accomplished using standard techniques known in the art appropriate to the host cell selected (e.g., electroporation, nuclear microinjection, bacterial protoplast fusion with intact cells, or polycations, e.g., polybrene, polyornithine, etc.).

A host cell is chosen, in various embodiments, that best suits the expression vector containing the components and allows for the most efficient and favorable production of the bispecific antibody species. Exemplary host cells for expression include those of prokaryotes and eukaryotes (single-cell or multiple-cell), bacterial cells (e.g., strains of E. coli, Bacillus spp., Streptomyces spp., etc.), mycobacteria cells, fungal cells, yeast cells (e.g., S. cerevisiae, S. pombe, P. pastoris, P. methanolica, etc.), plant cells, insect cells (e.g., SF-9, SF-21, baculovirus-infected insect cells, Trichoplusia ni, etc.), non-human animal cells, human cells, or cell fusions such as, for example, hybridomas or quadromas. In various embodiments, the cell is a human, monkey, ape, hamster, rat, or mouse cell. In various embodiments, the cell is a eukaryotic cell selected from CHO (e.g., CHO K1, DXB-11 CHO, Veggie-CHO), COS (e.g., COS-7), retinal cell, Vero, CV1, kidney (e.g., HEK293, 293 EBNA, MSR293, MDCK, HaK, BHK), HeLa, HepG2, WI38, MRC 5, Col0205, HB 8065, HL-60, (e.g., BHK21), Jurkat, Daudi, A431 (epidermal), CV-1, U937, 3T3, L cell, C127 cell, SP2/0, NS-0, MMT 060562, Sertoli cell, BRL 3A cell, HT1080 cell, myeloma cell, tumor cell, and a cell line derived from an aforementioned cell. In various embodiments, the cell comprises one or more viral genes, e.g. a retinal cell that expresses a viral gene (e.g., aPER.C6™ cell).

Mammalian host cells used to produce a multi-specific antigen-binding polypeptide, such as, e.g., a bispecific antibody, may be cultured in a variety of media. Commercially available media such as Ham's F10 (Sigma), Minimal Essential Medium ((MEM), Sigma), RPMI-1640 (Sigma), and Dulbecco's Modified Eagle's Medium ((DMEM), Sigma) are suitable for culturing the host cells. Media may be supplemented as necessary with hormones and/or other growth factors (such as insulin, transferrin, or epidermal growth factor), salts (such as sodium chloride, calcium, magnesium, and phosphate), buffers (such as HEPES), nucleosides (such as adenosine and thymidine), antibiotics (such as GENTAMYCIN), trace elements (defined as inorganic compounds usually present at final concentrations in the micromolar range), and glucose or an equivalent energy source. Any other supplements may also be included at appropriate concentrations as known to those skilled in the art. The culture conditions, such as temperature, pH, and the like, are, in various embodiments, those previously used with the host cell selected for expression, and will be apparent to those skilled in the art. The multi-specific antigen-binding polypeptide, e.g., bispecific antibody, is in various embodiments recovered from the culture medium as a secreted polypeptide, although it also may be recovered from host cell lysate when directly produced without a secretory signal. If the multi-specific antigen-binding polypeptide, e.g., bispecific antibody, is membrane-bound, it can be released from the membrane using a suitable detergent solution (e.g., Triton-X 100). Preferably, the multi-specific antigen-binding polypeptide, e.g., bispecific antibody, described herein involves the use of a first immunoglobulin C_{H}3 domain and a second immunoglobulin C_{H}3 domain, wherein the first and second immunoglobulin C_{H}3 domains differ from one another by at least one amino acid, and wherein at least one amino acid difference reduces binding of the bispecific antibody to Protein A as compared to a bispecific antibody lacking the amino acid difference (see US 2010/0331527 A1; herein incorporated by reference). In one embodiment, the first immunoglobulin C_{H}3 domain binds Protein A and the second immunoglobulin C_{H}3 domain contains a mutation that reduces or abolishes Protein A binding such as an H95R modification as disclosed herein. The second C_{H}3 may further comprise a Y96F modification as set forth above. Further modifications that may be found within the second C_{H}3 include: D16E, L18M, N44S, K52N, V57M, and V82I (by IMGT; D356E, L358M, N384S, K392N, V397M, and V422I by EU) in the case of IgG1 antibodies; N44S, KS2N, and V82I (IMGT; N384S, K392N, and V422I by EU) in the case of IgG2 antibodies; and QI5R, N44S, K52N, V57M, R69K, E79Q, and V82I (by IMGT; Q355R, N384S, K392N, V397M, R409K, E419Q, and V422I by EU) in the case of IgG4 antibodies. Variations on the bispecific antibody format described above are contemplated within the scope of the present invention.

Because of the dual nature of multi-specific antigen-binding polypeptides, e.g., bispecific antibodies (i.e., may be specific for different epitopes of one polypeptide or may contain antigen-binding domains specific for more than one target polypeptide, see, e.g., Tutt et aI., 1991, J. Immunol. 147:60-69; Kufer et aI., 2004, Trends Biotechnol. 22:238-244), they offer many useful advantages for therapeutic application. For example, multi-specific antigen-binding polypeptides, e.g., the bispecific antibodies, can be used for redirected cytotoxicity (e.g., to kill tumor cells), as a vaccine adjuvant, for delivering thrombolytic agents to clots, for converting enzyme activated prodrugs at a target site (e.g., a tumor), for treating infectious diseases, targeting immune complexes to cell surface receptors, or for delivering immunotoxins to tumor cells.

Other exemplary multi-specific antigen-binding polypeptides, e.g., bispecific antibody formats, that can be used in the context of the present invention include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes, etc.), CrossMab, CrossFab, (SEED)body, leucine zipper, Duobody, IgG1/IgG2, dual acting Fab (DAF)-IgG, and Mab2 bispecific formats (see, e.g., Klein et al. 2012, mAbs 4:6, 1-11, and references cited therein, for a review of the foregoing formats). Multi-specific antigen-binding polypeptides, e.g., bispecific antibodies, can also be constructed using peptide/nucleic acid conjugation, e.g., wherein unnatural amino acids with orthogonal chemical reactivity are used to generate site-specific antibody-oligonucleotide conjugates which then self-assemble into multimeric complexes with defined composition, valency and geometry. (See, e.g., Kazane et al., J. Am. Chem. Soc. [Epub: Dec. 4, 2012]).

The multi-specific antigen-binding polypeptides, e.g., bispecific antibodies, described herein can also be used in several therapeutic and non-therapeutic and/or diagnostic assay methods, such as, enzyme immunoassays, two-site immunoassays, in vitro or in vivo immunodiagnosis of various diseases (e.g., cancer), competitive binding assays, direct and indirect sandwich assays, and immunoprecipitation assays. Other uses for the multi-specific antigen-binding polypeptides, e.g., bispecific antibodies, will be apparent to those skilled in the art.

The antigen-binding polypeptides and fragments thereof, including antibodies and antibody fragments, of the present invention encompass polypeptides having amino acid sequences that vary from those of the described antibodies, but that retain the ability to bind the cell-specific and target antigens of the present invention. Such variant polypeptides and polypeptide fragments, including antibodies and antibody fragments, comprise one or more additions, deletions, or substitutions of amino acids when compared to parent sequence, but exhibit biological activity that is essentially equivalent to that of the parent molecule. Likewise, the antigen-binding polypeptides, including antibody-encoding DNA sequences of the present invention, encompass sequences that comprise one or more additions, deletions, or substitutions of nucleotides when compared to the disclosed sequence, but that encode an antigen-binding polypeptide or fragment thereof, including an antibody or antibody fragment, that is essentially bioequivalent to an antibody or antibody fragment of the invention.

Two antigen-binding polypeptides, or antibodies, are considered bioequivalent if, for example, they are pharmaceutical equivalents or pharmaceutical alternatives whose rate and extent of absorption do not show a significant difference when administered at the same molar dose under similar experimental conditions, either single dose or multiple doses. Some antigen-binding polypeptides or antibodies will be considered equivalents or pharmaceutical alternatives if they are equivalent in the extent of their absorption but not in their rate of absorption and yet may be considered bioequivalent because such differences in the rate of absorption are intentional and are reflected in the labeling, are not essential to the attainment of effective body drug concentrations on, e.g., chronic use, and are considered medically insignificant for the particular drug product studied.

In one embodiment, two antigen-binding polypeptides are bioequivalent if there are no clinically meaningful differences in their safety, purity, or potency.

In another embodiment, two antigen-binding polypeptides are bioequivalent if a patient can be switched one or more times between the reference product and the biological product without an expected increase in the risk of adverse effects, including a clinically significant change in immunogenicity, or diminished effectiveness, as compared to continued therapy without such switching.

In a further embodiment, two antigen-binding polypeptides are bioequivalent if they both act by a common mechanism or mechanisms of action for the condition or conditions of use, to the extent that such mechanisms are known.

Bioequivalence may be demonstrated by in vivo and/or in vitro methods. Bioequivalence measures include, e.g., (a) an in vivo test in humans or other mammals, in which the concentration of the antibody or its metabolites is measured in blood, plasma, serum, or other biological fluid as a function of time; (b) an in vitro test that has been correlated with and is reasonably predictive of human in vivo bioavailability data; (c) an in vivo test in humans or other mammals in which the appropriate acute pharmacological effect of the antibody (or its target) is measured as a function of time; and (d) in a well-controlled clinical trial that establishes safety, efficacy, or bioavailability or bioequivalence of an antibody.

Bioequivalent variants of the antigen-binding polypeptides, including the antibodies of the invention, may be constructed by, for example, making various substitutions of residues or sequences or deleting terminal or internal residues or sequences not needed for biological activity. For example, cysteine residues not essential for biological activity can be deleted or replaced with other amino acids to prevent formation of unnecessary or incorrect intramolecular disulfide bridges upon renaturation. In other contexts, bioequivalent antigen-binding polypeptides, including antibodies, may include antigen-binding polypeptides, including antibody variants, comprising amino acid changes, which modify the glycosylation characteristics of the antigen-binding polypeptide, e.g., antibodies, e.g., mutations that eliminate or remove glycosylation.

In some embodiments, Fc-containing polypeptides can comprise modifications in immunoglobulin domains, including where the modifications affect one or more effector functions of the binding polypeptide (e.g., modifications that affect FcγR binding, FcRn binding and thus half-life, and/or CDC activity). Such modifications include, but are not limited to, the following modifications and combinations thereof, with reference to EU numbering of an immunoglobulin constant region: 238, 239, 248, 249, 250, 252, 254, 255, 256, 258, 265, 267, 268, 269, 270, 272, 276, 278, 280, 283, 285, 286, 289, 290, 292, 293, 294, 295, 296, 297, 298, 301, 303, 305, 307, 308, 309, 311, 312, 315, 318, 320, 322, 324, 326, 327, 328, 329, 330, 331, 332, 333, 334, 335, 337, 338, 339, 340, 342, 344, 356, 358, 359, 360, 361, 362, 373, 375, 376, 378, 380, 382, 383, 384, 386, 388, 389, 398, 414, 416, 419, 428, 430, 433, 434, 435, 437, 438, and 439.

In one embodiment, the heavy chain constant region sequence comprises a modification in a C_{H}2 or a C_{H}3 region, wherein the modification increases affinity of the heavy chain constant region amino acid sequence to FcRn in an acidic environment (e.g., in an endosome where pH ranges from about 5.5 to about 6.0).

In another embodiment, the heavy chain constant region nucleotide sequence encodes a human heavy chain constant region amino acid sequence comprising a modification at position 250 (e.g., E or Q); 250 and 428 (e.g., L or F); 252 (e.g., L/Y/F/W or T); 254 (e.g., S or T); and 256 (e.g., S/R/Q/E/D or T); or a modification at position 428 and/or 433 (e.g., L/R/S/P/Q or K) and/or 434 (e.g., H/F or Y); or a modification at position 250 and/or 428; or a modification at position 307 or 308 (e.g., 308F, V308F), and 434. In a further embodiment, the modification comprises a 428L (e.g., M428L) and 434S (e.g., N434S) modification; a 428L, 259I (e.g., V259I), and 308F (e.g., V308F) modification; a 433K (e.g., H433K) and a 434 (e.g., 434Y) modification; a 252, 254, and 256 (e.g., 252Y, 254T, and 256E) modification; a 250Q and 428L modification (e.g., T250Q and M428L); and a 307 and/or 308 modification (e.g., 308F or 308P).

In another embodiment, the heavy chain constant region nucleotide sequence encodes a human C_{H}2 amino acid sequence comprising at least one modification between amino acid residues at positions 252 and 257, wherein the modification increases affinity of the human C_{H}2 amino acid sequence to FcRn in an acidic environment (e.g., in an endosome where pH ranges from about 5.5 to about 6.0).

In another embodiment, the heavy chain constant region nucleotide sequence encodes a human C_{H}2 amino acid sequence comprising at least one modification between amino acid residues at positions 307 and 311, wherein the modification increases affinity of the C_{H}2 amino acid sequence to FcRn in an acidic environment (e.g., in an endosome where pH ranges from about 5.5 to about 6.0).

In another embodiment, the heavy chain constant region nucleotide sequence encodes a human C_{H}3 amino acid sequence, wherein the C_{H}3 amino acid sequence comprises at least one modification between amino acid residues at positions 433 and 436, wherein the modification increases affinity of the C_{H}3 amino acid sequence to FcRn in an acidic environment (e.g., in an endosome where pH ranges from about 5.5 to about 6.0).

In another embodiment, the heavy chain constant region nucleotide sequence encodes a human heavy chain constant region amino acid sequence comprising a mutation selected from the group consisting of M428L, N434S, and a combination thereof.

In another embodiment, the heavy chain constant region nucleotide sequence encodes a human heavy chain constant region amino acid sequence comprising a mutation selected from the group consisting of M428L, V259I, V308F, and a combination thereof.

In another embodiment, the heavy chain constant region nucleotide sequence encodes a human heavy chain constant region amino acid sequence comprising an N434A mutation.

In another embodiment, the heavy chain constant region nucleotide sequence encodes a human heavy chain constant region amino acid sequence comprising a mutation selected from the group consisting of M252Y, S254T, T256E, and a combination thereof.

In another embodiment, the heavy chain constant region nucleotide sequence encodes a human heavy chain constant region amino acid sequence comprising a mutation selected from the group consisting of T250Q, M248L, or a combination thereof.

In another embodiment, the heavy chain constant region nucleotide sequence encodes a human heavy chain constant region amino acid sequence comprising a mutation selected from the group consisting of H433K, N434Y, or a combination thereof.

In another embodiment, the heavy chain constant region nucleotide sequence encodes a human heavy chain constant region amino acid sequence comprising a mutation selected from the group consisting of H433K, N434F, or a combination thereof.

In general, the antigen-binding polypeptides, including the antibodies of the present invention, function by binding to cell-specific or target antigens. The present invention includes antibodies to cell-specific or target antigens and antigen-binding fragments thereof that bind soluble monomeric, dimeric, or multimeric cell-specific or target antigen molecules with high affinity. The terms “antigen-binding fragment” of an antibody, or “antibody fragment”, as used herein, refers to one or more fragments of an antibody that retain the ability to bind to the cell-specific or target antigens of the present invention. For example, the present invention includes antigen-binding polypeptides, including the antibodies and antigen-binding fragments of antibodies that bind monomeric cell-specific or target antigens of the present invention (e.g., at 25° C. or at 37° C.) with a K_{D} of less than about 50 nM as measured by surface plasmon resonance. In certain embodiments, the antigen-binding polypeptides, including the antibodies or antigen-binding fragments thereof bind monomeric cell-specific or target antigens of the present invention with a K_{D} of less than about 40 nM, less than about 30 nM, less than about 20 nM, less than about 10 nM less than about 5 nM, less than about 2 nM or less than about 1 nM, as measured by surface plasmon resonance or a substantially similar assay.

The present invention also includes antigen-binding polypeptides and fragments thereof, such as, e.g., antibodies and antigen-binding fragments thereof that bind dimeric cell-specific or target antigens (e.g., at 25° C. or at 37° C.) with a K_{D} of less than about 400 pM as measured by surface plasmon resonance. In certain embodiments, the antigen-binding polypeptides and fragments thereof, such as, e.g., antibodies or antigen-binding fragments thereof, bind dimeric cell-specific or target antigens with a K_{D} of less than about 300 pM, less than about 250 pM, less than about 200 pM, less than about 100 pM, or less than about 50 pM, as measured by surface plasmon resonance or a substantially similar assay.

The present invention also includes antigen-binding polypeptides and fragments thereof, such as, e.g., antibodies and antigen-binding fragments thereof that bind the cell-specific or target antigens with a dissociative half-life (t½) of greater than about 1.1 minutes as measured by surface plasmon resonance at 25° C. or 37° C., or a substantially similar assay. In certain embodiments, the antigen-binding polypeptides and fragments thereof, such as, e.g., antibodies or antigen-binding fragments of the present invention bind CD3 with a t_{1/2} of greater than about 5 minutes, greater than about 10 minutes, greater than about 30 minutes, greater than about 50 minutes, greater than about 60 minutes, greater than about 70 minutes, greater than about 80 minutes, greater than about 90 minutes, greater than about 100 minutes, greater than about 200 minutes, greater than about 300 minutes, greater than about 400 minutes, greater than about 500 minutes, greater than about 600 minutes, greater than about 700 minutes, greater than about 800 minutes, greater than about 900 minutes, greater than about 1000 minutes, or greater than about 1200 minutes, as measured by surface plasmon resonance at 25° C. or 37° C. or a substantially similar assay.

In some embodiments, the antigen-binding polypeptides, e.g., the antibodies, of the present invention may bind to the extracellular domain of a cell-specific or target antigen of the present invention or to a particular region of the domain. In some embodiments, the antigen-binding polypeptides, such as, e.g., the antibodies, of the present invention may bind to more than one domain (cross-reactive antibodies).

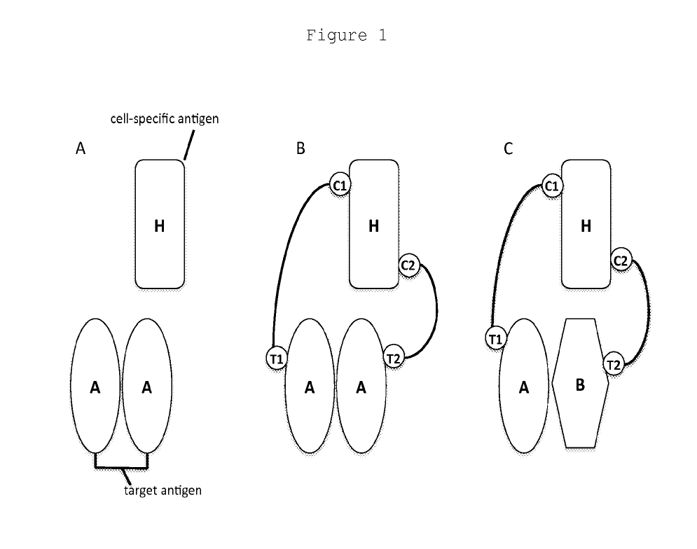

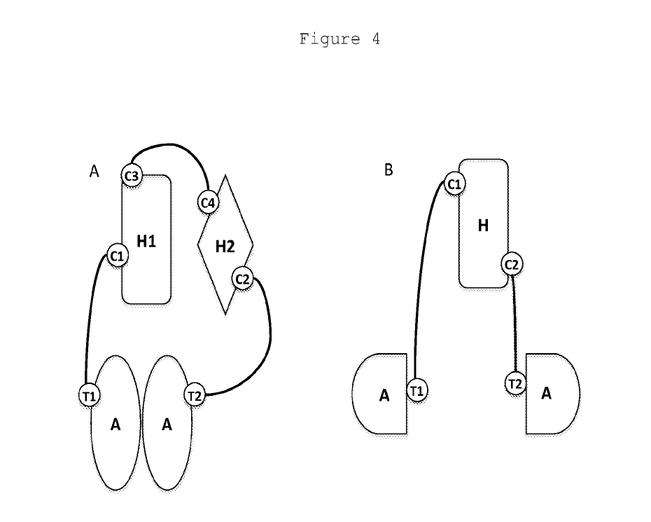

In certain embodiments, the antigen-binding polypeptides, e.g., the antibodies, of the present invention may function by blocking or inhibiting the activity of the target antigen. For example, the antigen-binding polypeptides may prevent the subunits of a dimeric target antigen from forming a complex, which complex is necessary for activation. See, e.g., FIG. 4B. In certain other embodiments, the antigen-binding polypeptides, e.g., the antibodies, may function by activating the target antigen. For example, the antigen-binding polypeptides may bring the subunits of a dimeric target antigen together allowing them to form a complex, which complex is necessary for activation. See, e.g., FIGS. 1B and 1C.

The antigen-binding polypeptides, e.g., antibodies, of the present invention may possess one or more of the aforementioned biological characteristics, or any combinations thereof. Other biological characteristics of the antigen-binding polypeptides, e.g., antibodies, of the present invention will be evident to a person of ordinary skill in the art from a review of the present disclosure including the working Examples herein.

The present invention encompasses human antigen-binding polypeptides, including monoclonal antibodies and antibody-derived polypeptides to cell-specific and target antigens conjugated to a therapeutic moiety (“immunoconjugate”), such as a cytotoxin or a chemotherapeutic agent to treat cancer. As used herein, the term “immunoconjugate” refers to an antibody which is chemically or biologically linked to a cytotoxin, a radioactive agent, a cytokine, an interferon, a target or reporter moiety, an enzyme, a toxin, a peptide or protein or a therapeutic agent. The antigen-binding polypeptides, e.g., antibody, may be linked to the cytotoxin, radioactive agent, cytokine, interferon, target or reporter moiety, enzyme, toxin, peptide or therapeutic agent at any location along the molecule so long as it is able to bind its target. Examples of immunoconjugates include antibody drug conjugates and antibody-toxin fusion proteins. In one embodiment, the agent may be a second different antibody to the cell-specific or target antigens of the present invention. In certain embodiments, the antigen-binding polypeptide, e.g., antibody, may be conjugated to an agent specific for a tumor cell or a virally infected cell. The type of therapeutic moiety that may be conjugated to the antigen-binding polypeptides, e.g., antibodies, of the present invention will take into account the condition to be treated and the desired therapeutic effect to be achieved. Examples of suitable agents for forming immunoconjugates are known in the art; see for example, WO 05/103081.

Variable regions of antibodies are typically isolated as single-chain Fv (scFv) or Fab fragments. ScFv fragments are composed of V_{H} and V_{L} domains linked by a short 10-25 amino acid linker. Once isolated, scFv fragments can be genetically linked with a flexible peptide linker such as, for example, one or more repeats of Ala-Ala-Ala, Gly-Gly-Gly-Gly-Ser, etc. The resultant peptide, a tandem scFv (taFv or scFv₂) can be arranged in various ways, with V_{H}-V_{L} or V_{L}—V_{H} ordering for each scFv of the taFv. (Kontermann, R. E. In: Bispecific Antibodies. Kontermann R E (ed.), Springer Heidelberg Dordrecht London New York, pp. 1-28 (2011)).

Bispecific diabodies (dAbs) are another form of antibody fragment and are within the scope of the present invention. In contrast to taFvs, diabodies are composed of two separate polypeptide chains from, for example, antibodies A and B, each chain bearing two variable domains (V_{H}A-V_{L} B and V_{H} B—V_{L}A or V_{L}A-V_{H} B and V_{L} B—V_{H}A). The linkers joining the variable domains are short (about five amino acids), preventing the association of V_{H} and V_{L} domains on the same chain, and promoting the association of V_{H} and V_{L} domains on different chains. Heterodimers that form are functional against both target antigens, (such as, e.g., V_{H}A-V_{L} B with V_{H} B—V_{L}A or V_{L}A-V_{H} B with V_{L} B—V_{H}A), however, homodimers can also form (such as, e.g., V_{H}A-V_{L} B with V_{H}A-V_{L} B, V_{H} B—V_{L}A with V_{H} B—V_{L}A, etc.), leading to nonfunctional molecules. Several strategies exist to prevent homodimerization, including the introduction of disulfide bonds to covalently join the two polypeptide chains, modification of the polypeptide chains to include large amino acids on one chain and small amino acids on the other (knobs-into-holes structures, discussed below), and addition of cysteine residues at C-terminal extensions. Another strategy is to join the two polypeptide chains by a linker sequence, producing a single-chain diabody molecule (scDb) that exhibits a more compact structure than a taFv. ScDbs or Dbs can be also be fused to the IgG1 C_{H}3 domain or the Fc region, producing di-diabodies. Examples of di-diabodies include, but are not limited to, scDb-Fc, Db-Fc, scDb-C_{H}3, and Db—C_{H}3. Additionally, scDbs can be used to make tetravalent bispecific molecules. By shortening the linker sequence of scDbs from about 15 amino acids to about 5 amino acids, dimeric single-chain diabody molecules result, known as TandAbs (Muller, D. and Kontermann, R. E. In: Bispecific Antibodies. Kontermann R E (ed.), Springer Heidelberg Dordrecht London New York, pp. 83-100 (2011)).

The terms “V_{H}/C_{H}1” and “V_{L}/C_{L}” refer to antibody fragment constructs comprising V_{H} and C_{H}1 domains, and V_{L} and C_{L} domains, respectively, as described above.

The following examples are provided to further illustrate the methods of the present invention. These examples are illustrative only and are not intended to limit the scope of the invention in any way.

### EXAMPLES

### Example 1

### Cell-Specific, Ligand-Independent Activation of FcER1a Receptor

The term “FcER1a” refers to a Fc fragment of IgE, high affinity I, receptor for alpha polypeptide. Examples of amino acid sequences for FcER1a and nucleic acid sequences that encode FcER1a (SEQ ID NOs 1-7) are shown in Table 1 below.

TABLE 1

| SEQ | Gene/ | | Nucleic | |
|---|---|---|---|---|
| ID | Protein | | Acid/ | Other |
| NO. | Name | Organism | Polypeptide | Information |
| 1 | FcER1a | Homo sapiens | Nucleic acid | |
| 2 | FcER1a | Mus musculus | Nucleic acid | |
| 3 | FcER1a | Rattus norvegicus | Nucleic acid | |
| 4 | FcER1a | Homo sapiens | Polypeptide | |
| 5 | FcER1a | Mus musculus | Polypeptide | |
| 6 | FcER1a | Rattus norvegicus | Polypeptide | Isoform CRA_a |
| 7 | FcER1a | Rattus norvegicus | Polypeptide | Isoform CRA_b |

FcER1a is expressed in mast cells, basophils, dendritic cells, Langerhans cells and monocytes. On these cells, FcER1 has a heterotetrameric form consisting of an alpha subunit, a beta subunit, and two gamma subunits. In humans, the trimeric form of the receptor also exists, with one alpha and two gamma subunits. The alpha subunit is responsible for IgE binding. It has two extracellular Ig-like domains, a transmembrane hydrophobic region, and a positively-charged cytoplasmic tail. (Sanak, et al., 2007).

The term “PD-1” refers to programmed death-1 (also called CD279). Examples of amino acid sequences for PD-1 and nucleic acid sequences that encode PD-1 (SEQ ID NOs. 8-13) are shown in Table 2 below.

TABLE 2

| SEQ | Gene/ | | Nucleic |
|---|---|---|---|
| ID | Protein | | acid/ |
| NO. | Name | Organism | polypeptide |
| 8 | PD-1 | Homo sapiens | Nucleic acid |
| 9 | PD-1 | Mus musculus | Nucleic acid |
| 10 | PD-1 | Rattus norvegicus | Nucleic acid |
| 11 | PD-1 | Homo sapiens | Polypeptide |
| 12 | PD-1 | Mus musculus | Polypeptide |
| 13 | PD-1 | Rattus norvegicus | Polypeptide |

PD-1 is a 288 amino acid protein receptor expressed on activated T-cells and B-cells, natural killer cells and monocytes. PD-1 is a member of the CD28/CTLA-4 (cytotoxic T lymphocyte antigen)/ICOS (inducible co-stimulator) family of T-cell co-inhibitory receptors (Chen et al 2013, Nature Rev. Immunol. 13: 227-242). The primary function of PD-1 is to attenuate the immune response (Riley 2009, Immunol. Rev. 229: 114-125). PD-1 has two ligands, PD-ligand1 (PD-L1) and PD-L2. PD-L1 (CD274, B7H1) is expressed widely on both lymphoid and non-lymphoid tissues such as CD4 and CD8 T-cells, macrophage lineage cells, peripheral tissues as well as on tumor cells, virally-infected cells and autoimmune tissue cells. PD-L2 (CD273, B7-DC) has a more restricted expression than PD-L1, being expressed on activated dendritic cells and macrophages (Dong et al 1999, Nature Med.). PD-1 binding to its ligands results in decreased T-cell proliferation and cytokine secretion, compromising humoral and cellular immune responses in diseases such as cancer, viral infection and autoimmune disease. Blockade of PD-1 binding to reverse immunosuppression has been studied in autoimmune, viral and tumor immunotherapy (Ribas 2012, NEJM 366: 2517-2519; Watanabe et al 2012, Clin. Dev. Immunol. Volume 2012, Article ID: 269756; Wang et al 2013, J. Viral Hep. 20: 27-39). PD1 is thought to exist as a monomer on the cell surface.

The term “CD300A” refers to cluster of differentiation 300A. Examples of amino acid sequences for CD300A and nucleic acid sequences that encode CD300A (SEQ ID NOs. 14-26) are shown in Table 3 below.

TABLE 3

| SEQ | Gene/ | | Nucleic | |
|---|---|---|---|---|
| ID | Protein | | acid/ | Other |
| NO. | Name | Organism | polypeptide | Information |
| 14 | CD300a | Homo sapiens | Nucleic acid | Variant 1 |
| 15 | CD300a | Homo sapiens | Nucleic acid | Variant 2 |
| 16 | CD300a | Mus musculus | Nucleic acid | mRNA |
| 17 | CD300a | Mus musculus | Nucleic acid | Complete CDS |
| 18 | CD300a | Rattus norvegicus | Nucleic acid | |
| 19 | CD300a | Homo sapiens | Polypeptide | |
| 20 | CD300a | Homo sapiens | Polypeptide | Isoform CRA_a |
| 21 | CD300a | Homo sapiens | Polypeptide | Isoform CRA_b |
| 22 | CD300a | Homo sapiens | Polypeptide | Isoform CRA_c |
| 23 | CD300a | Mus musculus | Polypeptide | |
| 24 | CD300a | Mus musculus | Polypeptide | Isoform CRA_a |
| 25 | CD300a | Mus musculus | Polypeptide | Isoform CRA_b |
| 26 | CD300a | Rattus norvegicus | Polypeptide | |

CD300A is member of the seven-gene CD300 family on human chromosome 17. CD300 molecules are members of the Ig super family bearing one Ig-like domain in their extracellular portions and can be found on myeloid cells, including macrophages, neutrophils, and/or mast cells, and may regulate the activation and inflammatory response of these cells. Additionally, CD300A is expressed on human natural killer cells and is involved in cytotoxic function. Upon cross-linking with monoclonal antibodies, CD300A, inhibits FcER1a-mediated signals, resulting in the suppression of degranulation from human and mouse mast cells in vitro. (Nakahashi-Oda, et al., 2012). It is not entirely clear if CD300A exists as a monomer or a higher order structure on the cell surface.

PD-1 or CD300a (with inactivated or deleted cytosolic domain), will be co-expressed with FcER1a in rat basophil leukemia (RBL) cells. The RBL cells also express luciferase linked to a nuclear factor of activated T-cells response element (NFAT-RE).

FcER1a antibody sequences are derived from universal light chain (ULC) sequences Vk1-39 (described previously in U.S. Patent Application No. 2013/0185821 A1, incorporated herein by reference) and heavy chain sequences derived from mAbs that activate RBL/FcER1a cells (SEQ ID NOs. 192-193 and 196-198, Table 4 below.

TABLE 4

| SEQ | | | Nucleic |
|---|---|---|---|
| ID | Antibody | | acid/ |
| NO. | fragment | Organism | polypeptide |
| 192 | anti-FcER1a_VH | Artificial sequence | Nucleic acid |
| 193 | anti-FcER1a_VH | Artificial sequence | Polypeptide |
| 196 | anti-FcER1a_HCDR1 | Artificial sequence | Polypeptide |
| 197 | anti-FcER1a_HCDR2 | Artificial sequence | Polypeptide |
| 198 | anti-FcER1a_HCDR3 | Artificial sequence | Polypeptide |

PD-1 and CD300A antibody sequences are derived from ULC sequences Vk1-39 and heavy chain sequences derived from parental PD-1 (SEQ ID NOs 202-203, 206-208, 212-213, and 216-218, Table 5 below) and CD300A mAbs. ULC mAbs that bind to PD-1 that do not cross-compete for binding to soluble PD-1 have been identified. Similar mAbs against CD300A have also been identified.

TABLE 5

| SEQ | | | Nucleic |
|---|---|---|---|
| ID | Antibody | | acid/ |
| No. | fragment | Organism | polypeptide |
| 202 | anti-PD-1_VH | Artificial sequence | Nucleic acid |
| 203 | anti-PD-1_VH | Artificial sequence | Polypeptide |
| 206 | anti-PD-1_HCDR1 | Artificial sequence | Polypeptide |
| 207 | anti-PD-1_HCDR2 | Artificial sequence | Polypeptide |
| 208 | anti-PD-1_HCDR3 | Artificial sequence | Polypeptide |
| 212 | anti-PD-1_VH | Artificial sequence | Nucleic acid |
| 213 | anti-PD-1_VH | Artificial sequence | Polypeptide |
| 216 | anti-PD-1_HCDR1 | Artificial sequence | Polypeptide |
| 217 | anti-PD-1_HCDR2 | Artificial sequence | Polypeptide |
| 218 | anti-PD-1_HCDR3 | Artificial sequence | Polypeptide |

The bispecific antibodies are made recombinantly. In the first construct, the nucleic acid encoding the V_{H} portion of an anti-FcER1a antibody (SEQ ID NO: 192 for example) is fused to the C_{H} portion of an IgG1. A second construct is similarly made by fusing the nucleic acid encoding the V_{H} portion of an anti-PD-1 antibody (SEQ ID NO: 202 for example) to the C_{H} portion of an IgG1. These two constructs, along with the ULC sequences (SEQ ID NO: 275, for example), are transfected into CHO cells and are co-expressed. The resulting antibodies are purified by a protein A column, followed by affinity purification with PD-1 and FcER1a.

To demonstrate cell-specific activation of FcER1a receptors, an NFAT-luciferase assay is utilized. RBL cells are co-transfected with FcER1a, PD-1 or CD300A, and NFAT-RE-linked luciferase. Upon the cell-specific activation of FcER1a, luciferase will be transcribed, and a signal will be detected. For PD-1, a construct with an inactivated immunoreceptor tyrosine-based switch motif (ITSM) cytosolic domain (Y/F mutant) is made and validated. For CD300A, a delta-cytosolic domain construct (or ectodomain with transmembrane domain, CD300A Ecto™) is generated. CD300A Ecto™ is transfected and selected for cell surface expression.

Activation is assessed using two bispecific Abs with target antigen binding domains that each bind to the same or overlapping epitopes on FcER1a (e.g., T1 and T2 as shown in FIG. 1B). The two bispecific mAbs will have two different cell-specific antigen binding domains that each bind to different epitopes on either PD-1 or CD300A (e.g., different C1 and C2). In these experiments, non-blocking mAbs may be required in order to allow for FcER1a expression on the cell surface. The combination of antibodies is incubated with the luciferase cell line above. It is expected that the combinations of the bispecific antibodies will be able to activate the FcER1a receptors (i.e., activate the transcription of luciferase).

The cell-specific activation of FcER1a receptor may strengthen the immune system's response to antigens and may be useful in cancer treatments.

### Example 2

### Cell-Specific, Ligand-Dependent Activation of TrkB Receptor

The term “TrkB” refers to TrkB tyrosine kinase or BDNF/NT-3 growth factor receptor or neurotrophic tyrosine kinase receptor, type 2. Examples of amino acid sequences for TrkB and nucleic acid sequences that encode TrkB (SEQ ID NOs. 27-41) are shown in Table 6 below.

TABLE 6

| SEQ | Gene/ | | Nucleic | |
|---|---|---|---|---|
| ID | Protein | | acid/ | Other |
| No. | Name | Organism | polypeptide | Information |
| 27 | TrkB | Homo sapiens | Nucleic acid | |
| 28 | TrkB | Homo sapiens | Nucleic acid | Alternatively spliced |
| 29 | TrkB | Mus musculus | Nucleic acid | Variant 1 |
| 30 | TrkB | Mus musculus | Nucleic acid | Variant 2 |
| 31 | TrkB | Mus musculus | Nucleic acid | Variant 3 |
| 32 | TrkB | Rattus norvegicus | Nucleic acid | Complete CDS |
| 33 | TrkB | Rattus norvegicus | Nucleic acid | Variant 1 |
| 34 | TrkB | Rattus norvegicus | Nucleic acid | Variant 2 |
| 35 | TrkB | Homo sapiens | Polypeptide | Accession: |
| | | | | AAB33109.1 |
| 36 | TrkB | Homo sapiens | Polypeptide | Accession: |
| | | | | AAB33110.1 |
| 37 | TrkB | Mus musculus | Polypeptide | |
| 38 | TrkB | Mus musculus | Polypeptide | Isoform a |
| 39 | TrkB | Mus musculus | Polypeptide | Isoform b |
| 40 | TrkB | Rattus norvegicus | Polypeptide | Isoform 1 |
| 41 | TrkB | Rattus norvegicus | Polypeptide | Isoform 2 |

The Trk family of receptors includes TrkA, TrkB and TrkC, and is instrumental in carrying out the cellular effects of neurotrophins. TrkB is a receptor for brain-derived neurotrophic factor (BDNF) and neurotrophin-4 (NT4) ligands. Examples of amino acid sequences for BDNF and nucleic acid sequences that encode BDNF (SEQ ID NOs 42-54) are shown in Table 7 below.

TABLE 7

| SEQ | Gene/ | | Nucleic | |
|---|---|---|---|---|
| ID | protein | | acid/ | Other |
| No. | name | Organism | polypeptide | Information |
| 42 | BDNF | Homo sapiens | Nucleic acid | Variant 1 |
| 43 | BDNF | Homo sapiens | Nucleic acid | Variant 2 |
| 44 | BDNF | Homo sapiens | Nucleic acid | Variant 3 |
| 45 | BDNF | Mus musculus | Nucleic acid | Variant 1 |
| 46 | BDNF | Mus musculus | Nucleic acid | Variant 2 |
| 47 | BDNF | Mus musculus | Nucleic acid | Variant 3 |
| 48 | BDNF | Rattus norvegicus | Nucleic acid | Variant 1 |
| 49 | BDNF | Rattus norvegicus | Nucleic acid | Variant 2 |
| 50 | BDNF | Rattus norvegicus | Nucleic acid | Variant 3 |
| 51 | BDNF | Homo sapiens | Polypeptide | |
| 52 | BDNF | Mus musculus | Polypeptide | Isoform 1 |
| 53 | BDNF | Mus musculus | Polypeptide | Isoform 2 |
| 54 | BDNF | Rattus norvegicus | Polypeptide | |

Tyrosine kinase receptors activate upon contact with the neurotrophin ligand and can dimerize with monomers that are not bound to a ligand. The dimeric and monomeric forms are believed to be in equilibrium, which may be critical to regulate downstream signaling pathways. TrkB is a type 1 membrane protein and may be incorporated in endosomes upon ligand binding. TrkB contains a protein kinase domain, two leucine rich repeats and two Ig-like C2 set domains, and is expressed in both the central (CNS) and peripheral nervous systems (PNS). In the CNS, a high TrkB expression is observed in cerebral cortex, hippocampus, thalamus, choroid plexus, and granular layer of the cerebellum, brain stem, retina and the spinal cord. In the PNS, it is expressed in the cranial ganglia, vestibular system, sub-maxillary glands and the dorsal root ganglia. TrkB is also expressed in the fetal brain and in a variety of other tissues like skeletal muscles, kidneys and pancreas. (Gupta, et al., 2013).

The term “Her2” refers to human epidermal growth factor receptor 2. Examples of amino acid sequences for Her2 and nucleic acid sequences that encode Her2 (SEQ ID NOs 55 67) are shown in Table 8 below.

TABLE 8

| SEQ | Gene/ | | Nucleic | |
|---|---|---|---|---|
| ID | protein | | acid/ | Other |
| No. | name | Organism | polypeptide | Information |
| 55 | Her2/ERBB2 | Homo sapiens | Nucleic acid | Variant 1 |
| 56 | Her2/ERBB2 | Homo sapiens | Nucleic acid | Variant 2 |
| 57 | Her2/ERBB2 | Mus musculus | Nucleic acid | |
| 58 | Her2/ERBB2 | Rattus norvegicus | Nucleic acid | |
| 59 | Her2/ERBB2 | Homo sapiens | Polypeptide | Isoform CRA_a |
| 60 | Her2/ERBB2 | Homo sapiens | Polypeptide | Isoform CRA_b |
| 61 | Her2/ERBB2 | Homo sapiens | Polypeptide | Isoform CRA_c |
| 62 | Her2/ERBB2 | Mus musculus | Polypeptide | Accession: |
| | | | | AAH46811.1 |
| 63 | Her2/ERBB2 | Mus musculus | Polypeptide | Accession: |
| | | | | AAH27080.2 |
| 64 | Her2/ERBB2 | Mus musculus | Polypeptide | Accession: |
| | | | | NP_001003817.1 |
| 65 | Her2/ERBB2 | Rattus norvegicus | Polypeptide | |
| 66 | Her2/ERBB2 | Rattus norvegicus | Polypeptide | Isoform CRA_a |
| 67 | Her2/ERBB2 | Rattus norvegicus | Polypeptide | Isoform CRA_b |

HER2/erbB-2 belongs to a family of four transmembrane receptors involved in signal transduction pathways that regulate cell growth and differentiation. HER2 mediates signaling to cancer cells, causing them to proliferate. HER receptors exist as monomers but dimerize upon ligand binding. Dimers typically consist of HER2 and HER3, the latter of which has no inherent activity. Overexpression of HER2 leads to more HER2-containing heterodimers, resulting in enhanced responsiveness to stromal growth factors and oncogenic transformation. (Yarden, et al., 2001).

The term “PSMA” refers to prostate-specific membrane antigen (PSMA). Examples of amino acid sequences for PSMA and nucleic acid sequences that encode PSMA (SEQ ID NOs. 68-84) are shown in Table 9 below.

TABLE 9

| SEQ | Gene/ | | Nucleic | |
|---|---|---|---|---|
| ID | protein | | acid/ | Other |
| No. | name | Organism | polypeptide | Information |
| 68 | PSMA | Homo sapiens | Nucleic acid | Variant 1 |
| 69 | PSMA | Homo sapiens | Nucleic acid | Variant 2 |
| 70 | PSMA | Homo sapiens | Nucleic acid | Variant 3 |
| 71 | PSMA | Homo sapiens | Nucleic acid | Variant 4 |
| 72 | PSMA | Homo sapiens | Nucleic acid | Variant 5 |
| 73 | PSMA | Mus musculus | Nucleic acid | Variant 1 |
| 74 | PSMA | Mus musculus | Nucleic acid | Variant 2 |
| 75 | PSMA | Rattus norvegicus | Nucleic acid | |
| 76 | PSMA | Homo sapiens | Polypeptide | Isoform 1 |
| 77 | PSMA | Homo sapiens | Polypeptide | Isoform 2 |
| 78 | PSMA | Homo sapiens | Polypeptide | Isoform 3 |
| 79 | PSMA | Homo sapiens | Polypeptide | Isoform 4 |
| 80 | PSMA | Homo sapiens | Polypeptide | Isoform 5 |
| 81 | PSMA | Mus musculus | Polypeptide | Isoform 1 |
| 82 | PSMA | Mus musculus | Polypeptide | Isoform 2 |
| 83 | PSMA | Rattus norvegicus | Polypeptide | Accession: |
| | | | | AAC40067.1 |
| 84 | PSMA | Rattus norvegicus | Polypeptide | Accession: |
| | | | | AAB96759.1 |

PSMA is a type II membrane protein that is highly expressed in prostatic intraepithelial neoplasia and in primary and metastatic prostate cancers. PSMA expression is also higher in prostate cancer cells from hormone-refractory patients. PSMA may also be a biomarker for disease recurrence. (Yao, et al., 2009). PSMA is thought to exist primarily as a homodimer on the cell surface.

Her2 or PSMA will be co-expressed with TrkB in human embryonic kidney 293 (HEK293) cells. The HEK293 cells also express luciferase linked to a serum response element (SRE-luciferase).

TrkB antibody sequences are derived from ULC sequences and heavy chain sequences derived from parental bivalent TrkB mAbs (SEQ ID NOs 222, 224-226, 230, and 232-234, Table 10 below). Activating or blocking mAbs, as well as non-blocking mAbs, may be used. Additionally, cross competition data may be gathered for TrkB antibody sequences.

TABLE 10

| SEQ | | | Nucleic |
|---|---|---|---|
| ID | Antibody | | acid/ |
| No. | fragment | Organism | polypeptide |
| 222 | anti-TrkB_VH | Artificial sequence | Polypeptide |
| 224 | anti-TrkB_HCDR1 | Artificial sequence | Polypeptide |
| 225 | anti-TrkB_HCDR2 | Artificial sequence | Polypeptide |
| 226 | anti-TrkB_HCDR3 | Artificial sequence | Polypeptide |
| 230 | anti-TrkB_VH | Artificial sequence | Polypeptide |
| 232 | anti-TrkB_HCDR1 | Artificial sequence | Polypeptide |
| 233 | anti-TrkB_HCDR2 | Artificial sequence | Polypeptide |
| 234 | anti-TrkB_HCDR3 | Artificial sequence | Polypeptide |

Her2 and PSMA antibody sequences are derived from ULC sequences and heavy chain sequences are derived from parental Her2 (SEQ ID NOs. 238, 240-242, 246, and 248-250, Table 11 below) and PSMA mAbs (SEQ ID NO. 254-255, 258-260, 264-265, and 268-270, Table 12 below). Cross competition data may be gathered for Her2 and PSMA antibody sequences as well.

TABLE 11

| SEQ | | | Nucleic |
|---|---|---|---|
| ID | Antibody | | acid/ |
| No. | fragment | Organism | polypeptide |
| 238 | anti-Her2_VH | Artificial sequence | Polypeptide |
| 240 | anti_Her2_HCDR1 | Artificial sequence | Polypeptide |
| 241 | anti_Her2_HCDR2 | Artificial sequence | Polypeptide |
| 242 | anti_Her2_HCDR3 | Artificial sequence | Polypeptide |
| 246 | anti-Her2_VH | Artificial sequence | Polypeptide |
| 248 | anti_Her2_HCDR1 | Artificial sequence | Polypeptide |
| 249 | anti_Her2_HCDR2 | Artificial sequence | Polypeptide |
| 250 | anti_Her2_HCDR3 | Artificial sequence | Polypeptide |

TABLE 12

| SEQ | | | Nucleic |
|---|---|---|---|
| ID | Antibody | | acid/ |
| No. | fragment | Organism | polypeptide |
| 254 | anti-PSMA_VH | Artificial sequence | Nucleic acid |
| 255 | anti-PSMA_VH | Artificial sequence | Polypeptide |
| 258 | anti_PSMA_HCDR1 | Artificial sequence | Polypeptide |
| 259 | anti_PSMA_HCDR2 | Artificial sequence | Polypeptide |
| 260 | anti_PSMA_HCDR3 | Artificial sequence | Polypeptide |
| 264 | anti-PSMA_VH | Artificial sequence | Nucleic acid |
| 265 | anti-PSMA_VH | Artificial sequence | Polypeptide |
| 268 | anti_PSMA_HCDR1 | Artificial sequence | Polypeptide |
| 269 | anti_PSMA_HCDR2 | Artificial sequence | Polypeptide |
| 270 | anti_PSMA_HCDR3 | Artificial sequence | Polypeptide |

The bispecific antibodies are made recombinantly. In the first construct, the nucleic acid encoding the V_{H} portion of an anti-TrkB antibody (SEQ ID NO: 222 for example) is fused to the C_{H} portion of an IgG1. A second construct is similarly made by fusing the nucleic acid encoding the V_{H} portion of an anti-Her2 antibody (SEQ ID NO: 238 for example) or an anti PSMA antibody (SEQ ID NO: 254 for example) to the C_{H} portion of an IgG1. These two constructs, along with the ULC sequences (SEQ ID NO: 275 for example), are transfected into CHO cells and are co-expressed. The resulting antibodies are purified by a protein A column, followed by affinity purification with TrkB and the appropriate cell-specific antigen (Her2 or PSMA).

To demonstrate target cell-specific clustering of TrkB receptors, a SRE-luciferase assay is utilized. HEK293 cells are co-transfected with TrkB, Her2 or PSMA, and SRE-luciferase. Upon the cell-specific dimerization and activation of TrkB, luciferase will be transcribed, and a signal will be detected. For both Her2 and PSMA, cell surface-specific expression is selected for and cell surface density is measured.

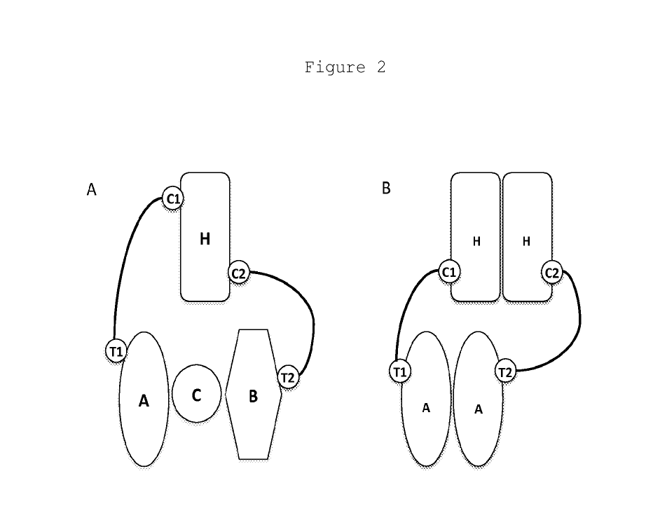

In the presence of a BDNF mimetic (as disclosed in e.g., Massa et al., 2010), activation is assessed using two bispecific Abs with target antigen binding domains that each bind to the same or overlapping epitopes on TrkB (e.g., T1 and T2 as shown in FIG. 2B). The two bispecific mAbs will have two different cell-specific antigen binding domains that each bind to different epitopes on Her2 (e.g., different C1 and C2). In these experiments, non-blocking mAbs may be required in order to allow for expression on the cell surface. The combination of antibodies is incubated with the luciferase cell line above. It is expected that the combinations of the bispecific antibodies will be able to activate the TrkB receptors (i.e., activate the transcription of luciferase).

In the presence of a BDNF mimetic (as disclosed in e.g., Massa et al., 2010), activation is assessed using bispecific Abs with target antigen binding domains that each bind to the same or overlapping epitopes on TrkB (e.g., T1 and T2 as shown in FIG. 2B). The bispecific mAbs will have two different or overlapping/same cell-specific antigen binding domains that each bind to different epitopes on PMSA (e.g., different or same C1 and C2). In these experiments, non-blocking mAbs may be required in order to allow for expression on the cell surface. The combination of antibodies is incubated with the luciferase cell line above. It is expected that the combinations of the bispecific antibodies will be able to activate the TrkB receptors (i.e., activate the transcription of luciferase).

The activation of TrkB in CNS neurons, such as retinal ganglion cells (RGCs), can be useful for promoting neuronal survival and neurite growth. The effect on RGC survival can be tested in multiple mouse models, such as, for example, an optic nerve injury model. (Tang et al., 2011). Activation of TrkB in brain could be useful during the recovery from brain injury, such as stroke.

### Example 3

### Cell-Specific, Ligand-Dependent Activation of FGFR1c

The term “FGF21” refers to fibroblast growth factor 21. Examples of amino acid sequences for FGF21 and nucleic acid sequences that encode FGF21 (SEQ ID NOs. 85-91) are shown in Table 13 below. FGF21 is a member of the FGF family which produces beneficial effects on lipid levels, body weight and glucose metabolism in animals.

TABLE 13

| SEQ | Gene/ | | Nucleic | |
|---|---|---|---|---|
| ID | protein | | acid/ | Other |
| No. | name | Organism | polypeptide | information |
| 85 | FGF21 | Homo sapiens | Nucleic acid | |
| 86 | FGF21 | Mus musculus | Nucleic acid | |
| 87 | FGF21 | Rattus norvegicus | Nucleic acid | |
| 88 | FGF21 | Homo sapiens | Polypeptide | Isoform CRA_1 |
| 89 | FGF21 | Homo sapiens | Polypeptide | Isoform CRA_2 |
| 90 | FGF21 | Mus musculus | Polypeptide | |
| 91 | FGF21 | Rattus norvegicus | Polypeptide | |

For example, overexpression of FGF21 in transgenic mice has been shown to result in reduced glucose and triglyceride levels, and resistance to diet-induced obesity. (Kharitonenkov et al. (2005), J. Clin. Invest. 115; 1627-1635). Moreover, the administration of exogenous FGF21 to rodents and primates results in normalization of plasma glucose levels, reduced triglyceride and cholesterol levels, improved glucose tolerance and improved insulin sensitivity. (Kharitonenkov et al. (2007), Endocrinol. 148:774-781) FGF21 administration in experimental animal models has been shown to reduce body weight and body fat by increasing energy expenditure, physical activity, and metabolic rate. (Long and Kharitonenkov (2011) Biochim. Biophys. Acta 1812:791-795). FGF21 signaling is mediated through its interaction with a receptor complex that includes βKlotho (KLB) and one of three different FGF receptors (FGFR1c, FGFR2c or FGFR3c). (Ogawa et al. (2007), Proc. Natl. Acad. Sci. USA 104:7432-7437; Suzuki et al. (2008), Mol. Endocrinol. 22:1006-1014). Examples of amino acid sequences for KLB and nucleic acid sequences that encode KLB (SEQ ID NOs. 92-96) are show in in Table 14 below. Examples of amino acid sequences for FGFR1c and nucleic acid sequences that encode FGFR1c (SEQ ID NOs. 97-110) are shown in Table 15 below.

TABLE 14

| SEQ | Gene/ | | Nucleic | |
|---|---|---|---|---|
| ID | protein | | acid/ | Other |
| No. | name | Organism | polypeptide | information |
| 92 | Beta Klotho | Homo sapiens | Nucleic acid | |
| 93 | Beta Klotho | Mus musculus | Nucleic acid | |
| 94 | Beta Klotho | Mus musculus | Nucleic acid | Variant 1 |
| 95 | Beta Klotho | Homo sapiens | Polypeptide | |
| 96 | Beta Klotho | Mus musculus | Polypeptide | |

TABLE 15

| SEQ | Gene/ | | Nucleic | |
|---|---|---|---|---|
| ID | protein | | acid/ | Other |
| No. | name | Organism | polypeptide | information |
| 97 | FGFR1 | Homo sapiens | Nucleic acid | Variant 1 |
| 98 | FGFR1 | Homo sapiens | Nucleic acid | Variant 2 |
| 99 | FGFR1 | Homo sapiens | Nucleic acid | Variant 3 |
| 100 | FGFR1 | Mus musculus | Nucleic acid | Variant 1 |
| 101 | FGFR1 | Mus musculus | Nucleic acid | Variant 2 |
| 102 | FGFR1 | Mus musculus | Nucleic acid | Variant 3 |
| 103 | FGFR1 | Rattus norvegicus | Nucleic acid | |
| 104 | FGFR1 | Homo sapiens | Polypeptide | Isoform 1 |
| 105 | FGFR1 | Homo sapiens | Polypeptide | Isoform 2 |
| 106 | FGFR1 | Homo sapiens | Polypeptide | Isoform 3 |
| 107 | FGFR1 | Mus musculus | Polypeptide | Isoform 1 |
| 108 | FGFR1 | Mus musculus | Polypeptide | Isoform 2 |
| 109 | FGFR1 | Mus musculus | Polypeptide | Isoform 3 |
| 110 | FGFR1 | Rattus norvegicus | Polypeptide | |

It is believed that the main functional receptor for FGF21 signaling in vivo is the KLB/FGFR1c heterodimeric complex. Pharmacological activation of FGF21 signaling has been proposed for the treatment of various diseases and disorders in humans including type-2 diabetes, obesity, dyslipidemia, and other metabolic conditions. Proposed therapeutic strategies for activating FGF21 signaling include administration of recombinant FGF21, and the use of agonistic antibodies that bind FGFR1 or the KLB/FGFR1c complex (US 2011/0135657; US 2012/0294861; WO 2011/130417).

The term “GCGR” refers to a glucagon receptor. Examples of amino acid sequences for GCGR and nucleic acid sequences that encode GCGR (SEQ ID NOs. 111-121) are shown in Table 16 below. Glucagon likely interacts with GCGR in a similar fashion to the interaction of other peptide ligands with class B GPCRs. (Koth, et al., 2012). GCGR is thought to exist primarily as a monomer on the cell surface.

TABLE 16

| | Gene/ | | | |
|---|---|---|---|---|
| SEQ ID | protein | | Nucleic | Other |
| No. | name | Organism | acid/polypeptide | information |
| 111 | GCGR | Homo sapiens | Nucleic acid | |
| 112 | GCGR | Mus musculus | Nucleic acid | |
| 113 | GCGR | Rattus | Nucleic acid | Variant 1 |
| | | norvegicus | | |
| 114 | GCGR | Rattus | Nucleic acid | Variant 2 |
| | | norvegicus | | |
| 115 | GCGR | Homo sapiens | Polypeptide | |
| 116 | GCGR | Mus musculus | Polypeptide | Isoform CRA_a |
| 117 | GCGR | Mus musculus | Polypeptide | Isoform CRA_b |
| 118 | GCGR | Mus musculus | Polypeptide | Isoform CRA_c |
| 119 | GCGR | Rattus | Polypeptide | |
| | | norvegicus | | |
| 120 | GCGR | Rattus | Polypeptide | Isoform CRA_a |
| | | norvegicus | | |
| 121 | GCGR | Rattus | Polypeptide | Isoform CRA_b |
| | | norvegicus | | |

FGFR1c antibody sequences are derived from scFvs to FGFR1c (SEQ ID NO. 330-333, shown in Table 17 below).

TABLE 17

| SEQ ID No. | Antibody fragment | Organism | Nucleic acid/polypeptide |
|---|---|---|---|
| 330 | anti-FGFR1c_VH | human | Polypeptide |
| 331 | anti-FGFR1c_VH | human | Nucleic acid |
| 332 | anti-FGFR1c_VH | human | Polypeptide |
| 333 | anti-FGFR1c_VH | human | Nucleic acid |

ScFv binding to FGFR1 is assessed and cross competition data is obtained. GCGR antibody sequences comprise both blockers and non-blockers. (SEQ ID NO. 274-275, 278-280, 284-285, and 288-290, Table 18 below) and cross competition data for each is obtained.

TABLE 18

| | | | Nucleic acid/ |
|---|---|---|---|
| SEQ ID No. | Antibody fragment | Organism | polypeptide |
| 274 | anti-GCGR_VH | Artificial sequence | Nucleic acid |
| 275 | anti-GCGR_VH | Artificial sequence | Polypeptide |
| 278 | anti-GCGR_HCDR1 | Artificial sequence | Polypeptide |
| 279 | anti-GCGR_HCDR2 | Artificial sequence | Polypeptide |
| 280 | anti-GCGR_HCDR3 | Artificial sequence | Polypeptide |
| 284 | anti-GCGR_VH | Artificial sequence | Nucleic acid |
| 285 | anti-GCGR_VH | Artificial sequence | Polypeptide |
| 288 | anti-GCGR_HCDR1 | Artificial sequence | Polypeptide |
| 289 | anti-GCGR_HCDR2 | Artificial sequence | Polypeptide |
| 290 | anti-GCGR_HCDR3 | Artificial sequence | Polypeptide |

The bispecific antibodies are made recombinantly. In the first construct, the nucleic acid encoding the V_{H} portion of an anti-FGFR1c antibody is fused to the C_{H} portion of an IgG1. A second construct is similarly made by fusing the nucleic acid encoding the V_{H} portion of an anti-GGCR antibody (SEQ ID NO: 274 for example) to the C_{H} portion of an IgG1. These two constructs, along with the ULC sequences (SEQ ID NO: 275 for example), are transfected into CHO cells and are co-expressed. The resulting antibodies are purified by a protein A column, followed by affinity purification with FGFR1c and GGCR.

To demonstrate target cell-specific clustering of FGFR1c receptors, an SRE-luciferase assay is utilized. HEK293 cells are co-transfected with FGFR1c, GCGR, KLB, and SRE-luciferase. For GCGR, cell surface-specific expression is selected for and receptor number is determined. Upon the cell-specific clustering of FcER1a, luciferase will be transcribed, and a signal will be detected.

In the presence of an FGF21 mimetic (as disclosed in, e.g., Foltz et al., 2012), activation is assessed using bispecific Abs with target antigen binding domains that each bind to the same or overlapping epitopes on FGFR1c (e.g., T1 and T2 as shown in FIG. 2A). The two bispecific mAbs will have two different cell-specific antigen binding domains that each bind to different or same/overlapping epitopes on either GCGR (e.g., same or different C1 and C2). In these experiments, non-blocking mAbs may be required in order to allow for FGFR1c expression on the cell surface. The combination of antibodies is incubated with the luciferase cell line above. It is expected that all combinations of the bispecific antibodies will be able to dimerize FGFR1c/KLB receptor complexes and activate the receptor (i.e., activate the transcription of luciferase).

The cell-specific activation of FGFR1c receptor in liver and kidney, where GCGR is expressed, may be useful to improve aspects of the metabolic syndrome and reduce body weight in obese and diabetic individuals.

### Example 4

### Cell-Specific, Ligand-Dependent Activation of FGFR1c Wherein the Cell-Specific Antigen is Part of the Receptor Complex

FGFR1c antibody sequences are derived from scFvs to FGFR1c (SEQ ID NO. 330-333, shown in Table 17 above).

ScFv binding to FGFR1c is assessed and cross competition data is obtained. KLB antibody sequences are derived from ULC sequences and heavy chain sequences derived from parental KLB mAbs.

The bispecific antibodies are made recombinantly. In the first construct, the nucleic acid encoding the V_{H} portion of an anti-FGFR1c antibody is fused to the C_{H} portion of an IgG1. A second construct is similarly made by fusing the nucleic acid encoding the V_{H} portion of an anti-KLB antibody to the C_{H} portion of an IgG1. These two constructs, along with the ULC sequences (SEQ ID NO: 275 for example), are transfected into CHO cells and are co-expressed. The resulting antibodies are purified by a protein A column, followed by affinity purification with FGFR1c and KLB.

To demonstrate cell-specific dimerization of FGFR1c/KLB receptors, an SRE-luciferase assay is utilized. HEK293 cells are co-transfected with FGFR1c, KLB, and SRE-luciferase. For GCGR, cell surface-specific expression is selected for and cell surface density is determined. Upon the cell-specific clustering of FcER1a, luciferase will be transcribed, and a signal will be detected.

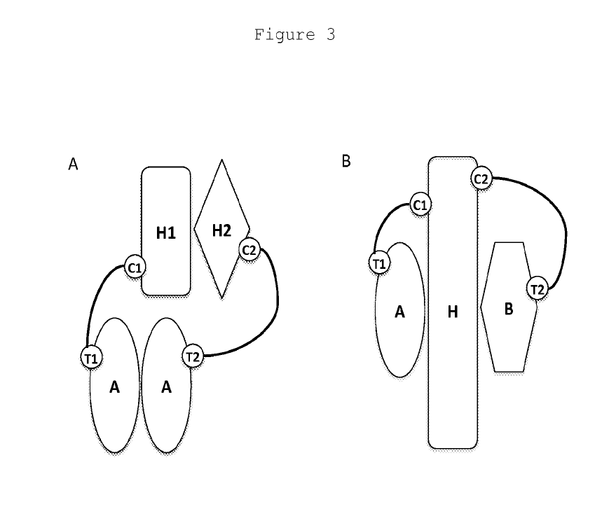

In the presence of an FGF21 mimetic (as disclosed in, e.g., Foltz et al., 2012), activation is assessed using two bispecific Abs with target antigen binding domains that each bind to the same or overlapping epitopes on FGFR1c (e.g., T1 and T2 as shown in FIG. 3B). The two bispecific mAbs will have two different cell-specific antigen binding domains that each bind to different epitopes on KLB (e.g., different C1 and C2). In these experiments, non-blocking mAbs may be required in order to allow for FGFR1c expression on the cell surface. The combination of antibodies is incubated with the luciferase cell line above. It is expected that all combinations of the bispecific antibodies will be able to dimerize FGFR1c/KLB receptor complexes and activate the receptor (i.e., activate the transcription of luciferase).

The cell-specific activation of FGFR1c receptor in hypothalamus, where may be useful in treatments related to disorders caused by hormone imbalances.

### Example 5

### Cell-Specific Activation of FGFR1c Wherein the Cell-Specific Antigen is Part of the Receptor Complex

Protein Y is expressed on adipocytes, liver and pancreas. Protein Y may be used to activate FGR1c in the presence of an FGF21 mimetic. The following combination of bispecific antibodies is used: on one bispecific antibody, a protein Y epitope A-binding domain and an FGFR1c epitope X-binding domain, and on the other bispecific antibody, a protein Y epitope B-binding domain (distinct from epitope A) and an FGFR1c epitope X-binding domain (same as the epitope for the previous bispecific antibody). Using this combination of bispecific antibodies in the presence of a FGF21 mimetic, FGFR1c is specifically activated in adipocytes, liver and pancreas.

The cell-specific activation of FGFR1c receptor in adipocytes, liver and pancreas may be useful to improve aspects of the metabolic syndrome and reduce body weight in obese and diabetic individuals.

### Example 6

### Cell-Specific, Heterologous Activation of FGFR1c Wherein the Cell-Specific Antigen is Part of the Receptor Complex

The term “CNTFRa” refers to ciliary neurotrophic factor receptor a. Examples of amino acid sequences for CNTFa and nucleic acid sequences that encode CNTFRa (SEQ ID NOs. 122-133) are shown in Table 19 below.

TABLE 19

| | Gene/ | | | |
|---|---|---|---|---|
| SEQ ID | protein | | Nucleic | Other |
| No. | name | Organism | acid/polypeptide | information |
| 122 | CNTFR | Homo sapiens | Nucleic acid | Variant 1 |
| 123 | CNTFR | Homo sapiens | Nucleic acid | Variant 2 |
| 124 | CNTFR | Homo sapiens | Nucleic acid | Variant 3 |
| 125 | CNTFR | Mus musculus | Nucleic acid | Variant 1 |
| 126 | CNTFR | Mus musculus | Nucleic acid | Variant 2 |
| 127 | CNTFR | Mus musculus | Nucleic acid | Variant 3 |
| 128 | CNTFR | Rattus | Nucleic acid | |
| | | norvegicus | | |
| 129 | CNTFR | Homo sapiens | Polypeptide | Isoform CRA_a |
| 130 | CNTFR | Mus musculus | Polypeptide | Isoform 1 |
| 131 | CNTFR | Mus musculus | Polypeptide | Isoform 2 |
| 132 | CNTFR | Rattus | Polypeptide | Isoform CRA_a |
| | | norvegicus | | |
| 133 | CNTFR | Rattus | Polypeptide | Isoform CRA_b |
| | | norvegicus | | |

The CNTF receptor complex is most closely related to the receptor complexes for interleukin-6 and leukemia inhibitory factor. CNTFRa is responsible for the specificity of the receptor and is expressed mainly in the nervous system and skeletal muscle. Signal transduction by CNTF requires that it bind first to CNTFRa, permitting the recruitment of glycoprotein 130 (gp130) and leukemia inhibitory factor receptor b (LIFR b), forming a heterotrimer receptor complex. In addition to its neuronal actions, CNTF and its analogs act on other cell types such as glia, hepatocytes, skeletal muscle, embryonic stem cells and bone marrow stromal cells (Sleeman, et al., 2000). Examples of amino acid sequences for gp130 and nucleic acid sequences that encode gp130 (SEQ ID NOs 334-339) are shown in Table 20 below. Examples of amino acid sequences for LIFR b and nucleic acid sequences that encode LIFR b (SEQ ID NOs 340-341) are shown in Table 21 below.

TABLE 20

| | Gene/Protein | | |
|---|---|---|---|
| SEQ ID No. | Name | Organism | Nucleic acid/polypeptide |
| 334 | gp130 | human | Polypeptide |
| 335 | gp130 | human | Nucleic acid |
| 336 | gp130 | mouse | Polypeptide |
| 337 | gp130 | mouse | Nucleic acid |
| 338 | gp130 | Rattus norvegicus | Polypeptide |
| 339 | gp130 | Rattus norvegicus | Nucleic acid |

TABLE 21

| SEQ ID No. | Gene/Protein Name | Organism | Nucleic acid/polypeptide |
|---|---|---|---|
| 340 | LIFR b | human | Polypeptide |
| 341 | LIFR b | human | Nucleic acid |

A bispecific antibody is made which includes a CNTFRa epitope A-binding domain and a gp130-binding domain. The other bispecific antibody includes a CNTFRa epitope B-binding domain (distinct from epitope A) and a LIFRb-binding domain. Using this combination of bispecific antibodies in the presence of a FGF21 mimetic, the heterotrimer of CNFRa, gp130, and LIFRb is formed.

The cell-specific activation of FGFR1c receptor in the nervous system, where CNTFRa is expressed, may be used to treat nervous system disorders.

### Example 7

### Cell-Specific, Ligand-Dependent, Heterologous Activation of IL4R and IL2Rgamma

Interleukin-4 (IL-4) is a cytokine produced by T helper cells, mast cells, and basophils. Examples of amino acid sequences for IL-4 and nucleic acid sequences that encode IL-4 (SEQ ID NOs. 134-141) are shown in Table 22 below.

TABLE 22

| SEQ ID | Gene/protein | | Nucleic | Other |
|---|---|---|---|---|
| No. | name | Organism | acid/polypeptide | information |
| 134 | IL-4 | Homo sapiens | Nucleic acid | Variant 1 |
| 135 | IL-4 | Mus musculus | Nucleic acid | Variant 1 |
| 136 | IL-4 | Rattus | Nucleic acid | |
| | | norvegicus | | |
| 137 | IL-4 | Homo sapiens | Polypeptide | Isoform 1 |
| | | | | precursor |
| 138 | IL-4 | Homo sapiens | Polypeptide | |
| 139 | IL-4 | Mus musculus | Polypeptide | Precursor |
| 140 | IL-4 | Mus musculus | Polypeptide | CAA28731.1 |
| 141 | IL-4 | Rattus | Polypeptide | |
| | | norvegicus | | |

IL-4 participates in growth stimulation of T cells, mast cells, granulocytes, megakaryocytes, and erythrocytes. IL-4 plays a critical role in the development of allergic diseases, and is most commonly associated with asthma, allergies, and diseases generally characterized by difficulty breathing. IL-4 binds to IL-4 receptor (IL-4R), an endogenous membrane-bound protein on the surface of certain cells. Examples of amino acid sequences for IL-4R and nucleic acid sequences that encode IL-4R (SEQ ID NOs. 142-151) are shown in Table 23 below. Upon binding of IL-4, IL-4R produces a signal that leads to clinical symptoms. Mature human IL4R has three domain structures: an extracellular domain, a membrane passage region, and an intracytoplasmic domain. (U.S. Pat. No. 7,449,201 B2).

TABLE 23

| | Gene/ | | | |
|---|---|---|---|---|
| SEQ ID | protein | | Nucleic | |
| No. | name | Organism | acid/polypeptide | Other information |
| 142 | IL-4R | Homo sapiens | Nucleic acid | Variant 1 |
| 143 | IL-4R | Homo sapiens | Nucleic acid | Variant 3 |
| 144 | IL-4R | Homo sapiens | Nucleic acid | Variant 4 |
| 145 | IL-4R | Homo sapiens | Nucleic acid | Variant 5 |
| 146 | IL-4R | Mus musculus | Nucleic acid | mRNA, complete |
| | | | | CDS |
| 147 | IL-4R | Rattus | Nucleic acid | NM_133380.2 |
| | | norvegicus | | |
| 148 | IL-4R | Rattus | Nucleic acid | X69903.1 |
| | | norvegicus | | |
| 149 | IL-4R | Homo sapiens | Polypeptide | |
| 150 | IL-4R | Mus musculus | Polypeptide | |
| 151 | IL-4R | Rattus | Polypeptide | |
| | | norvegicus | | |

The term “IL-2Rgamma” refers to interleukin-2 receptor gamma. Examples of amino acid sequences for IL-2Rgamma and nucleic acid sequences that encode IL-2Rgamma (SEQ ID NOs. 152-158) are shown in Table 24 below. IL-2Rgamma is a receptor subunit common to a number of interleukin receptors, including IL-2R and IL-4R.

Type I interferons (IFN) are a family of structurally related cytokines having antiviral, antitumor and immunomodulatory effects. The human IFNα (SEQ ID NOs. 159-164, shown in Table 25 below) locus includes two subfamilies.

TABLE 24

| | Gene/ | | | |
|---|---|---|---|---|
| SEQ ID | protein | | Nucleic | Other |
| No. | name | Organism | acid/polypeptide | information |
| 152 | IL-2Rg | Homo sapiens | Nucleic acid | |
| 153 | IL-2Rg | Mus musculus | Nucleic acid | |
| 154 | IL-2Rg | Rattus | Nucleic acid | |
| | | norvegicus | | |
| 155 | IL-2Rg | Homo sapiens | Polypeptide | |
| 156 | IL-2Rg | Mus musculus | Polypeptide | Isoform CRA_a |
| 157 | IL-2Rg | Mus musculus | Polypeptide | Isoform CRA_b |
| 158 | IL-2Rg | Rattus | Polypeptide | |
| | | norvegicus | | |

TABLE 25

| | | | Nucleic acid/ |
|---|---|---|---|
| SEQ ID NOs. | Gene/protein name | Organism | polypeptide |
| 159 | IFNA1 | Homo sapiens | Nucleic acid |
| 160 | IFNA1 | Mus musculus | Nucleic acid |
| 161 | IFNA1 | Rattus norvegicus | Nucleic acid |
| 162 | IFNA1 | Homo sapiens | Polypeptide |
| 163 | IFNA1 | Mus musculus | Polypeptide |
| 164 | IFNA1 | Rattus norvegicus | Polypeptide |

The subtypes of IFNα have different specific activities but they possess the same biological spectrum and have the same cellular receptor. The interferon β is encoded by a single gene which has approximately 50% homology with the IFNα genes (SEQ ID NOs. 165-170, shown in Table 26 below).

TABLE 26

| | | | Nucleic acid/ |
|---|---|---|---|
| SEQ ID NOs. | Gene/protein name | Organism | polypeptide |
| 165 | IFNB1 | Homo sapiens | Nucleic acid |
| 166 | IFNB1 | Mus musculus | Nucleic acid |
| 167 | IFNB1 | Rattus norvegicus | Nucleic acid |
| 168 | IFNB1 | Homo sapiens | Polypeptide |
| 169 | IFNB1 | Mus musculus | Polypeptide |
| 170 | IFNB1 | Rattus norvegicus | Polypeptide |

All human type I interferons bind to a cell surface receptor (IFN alpha receptor, IFNAR) consisting of two transmembrane proteins, IFNAR-1 and IFNAR-2. Examples of amino acid sequences for IFNAR-1 and nucleic acid sequences that encode IFNAR-1 (SEQ ID NOs 171-181) are shown in Table 27 below. Examples of amino acid sequences for IFNAR-2 and nucleic acid sequences that encode IFNAR-2 (SEQ ID NOs 182-191) are shown in Table 28 below. IFNAR-1 is essential for high affinity binding and differential specificity of the IFNAR complex. Each IFN subtype may produce diverse signaling effects upon interaction with the IFNAR complex. (U.S. Pat. No. 8,460,668 B2).

TABLE 27

| | Gene/ | | | |
|---|---|---|---|---|
| SEQ ID | protein | | Nucleic acid/ | Other |
| No. | name | Organism | polypeptide | information |
| 171 | IFNAR1 | Homo sapiens | Nucleic acid | |
| 172 | IFNAR1 | Mus musculus | Nucleic acid | |
| 173 | IFNAR1 | Rattus | Nucleic acid | Variant 1 |
| | | norvegicus | | |
| 174 | IFNAR1 | Rattus | Nucleic acid | Variant 2 |
| | | norvegicus | | |
| 175 | IFNAR1 | Homo sapiens | Polypeptide | Isoform CRA_a |
| 176 | IFNAR1 | Homo sapiens | Polypeptide | Isoform CRA_b |
| 177 | IFNAR1 | Mus musculus | Polypeptide | Isoform CRA_a |
| 178 | IFNAR1 | Mus musculus | Polypeptide | Isoform CRA_b |
| 179 | IFNAR1 | Mus musculus | Polypeptide | Isoform CRA_c |
| 180 | IFNAR1 | Rattus | Polypeptide | Isoform 1 |
| | | norvegicus | | |
| 181 | IFNAR1 | Rattus | Polypeptide | Isoform 2 |
| | | norvegicus | | |

TABLE 28

| SEQ ID | Gene/protein | | Nucleic | Other |
|---|---|---|---|---|
| No. | name | Organism | acid/polypeptide | information |
| 182 | IFNAR2 | Homo | Nucleic acid | Variant 1 |
| | | sapiens | | |
| 183 | IFNAR2 | Homo | Nucleic acid | Variant 2 |
| | | sapiens | | |
| 184 | IFNAR2 | Homo | Nucleic acid | Variant 3 |
| | | sapiens | | |
| 185 | IFNAR2 | Mus | Nucleic acid | Variant 1 |
| | | musculus | | |
| 186 | IFNAR2 | Mus | Nucleic acid | Variant 2 |
| | | musculus | | |
| 187 | IFNAR2 | Homo | Polypeptide | Isoform CRA_a |
| | | sapiens | | |
| 188 | IFNAR2 | Homo | Polypeptide | Isoform CRA_b |
| | | sapiens | | |
| 189 | IFNAR2 | Homo | Polypeptide | Isoform CRA_c |
| | | sapiens | | |
| 190 | IFNAR2 | Mus | Polypeptide | Isoform CRA_a |
| | | musculus | | |
| 191 | IFNAR2 | Mus | Polypeptide | Isoform CRA_b |
| | | musculus | | |

Her2 or PSMA is co-expressed with IL-4R and IL-2Rgamma in Ramos.2G5.4C10 cells. These cells also express luciferase linked to signal transducer and activator of transcription 3 (STAT3).

IL-4R antibody sequences are derived from ULC sequences and heavy chain sequences derived from parental bivalent IL-4R mAbs (SEQ ID NOs 314, 316-318, 322, 324-326, Table 29). Blocking mAbs, as well as non-blocking mAbs, may be used. Additionally, cross competition data may be gathered for IL-4R antibody sequences.

TABLE 29

| | | | Nucleic acid/ |
|---|---|---|---|
| SEQ ID Nos | Antibody fragment | Organism | polypeptide |
| 314 | anti-IL-4R_VH | Artificial sequence | Polypeptide |
| 316 | anti-IL-4R_HCDR1 | Artificial sequence | Polypeptide |
| 317 | anti-IL-4R_HCDR2 | Artificial sequence | Polypeptide |
| 318 | anti-IL-4R_HCDR3 | Artificial sequence | Polypeptide |
| 322 | anti-IL-4R_VH | Artificial sequence | Polypeptide |
| 324 | anti-IL-4R_HCDR1 | Artificial sequence | Polypeptide |
| 325 | anti-IL-4R_HCDR2 | Artificial sequence | Polypeptide |
| 326 | anti-IL-4R_HCDR3 | Artificial sequence | Polypeptide |

IL-2Rgamma antibody sequences are derived from ULC sequences and heavy chain sequences derived from parental bivalent IL-2Rgamma mAbs. Blocking mAbs, as well as non-blocking mAbs, may be used. Additionally, cross competition data may be gathered for IL-2Rgamma antibody sequences.

Her2 and PSMA antibody sequences are derived from ULC sequences and heavy chain sequences as discussed above.

The bispecific antibodies are made recombinantly. In the first construct, the nucleic acid encoding the V_{H} portion of an anti-IL4R antibody (SEQ ID NO: 314 for example) or an IL-2Rgamma antibody is fused to the C_{H} portion of an IgG1. A second construct is similarly made by fusing the nucleic acid encoding the V_{H} portion of an anti-Her2 antibody (SEQ ID NO: 238 for example) or an anti-PSMA antibody (SEQ ID NO: 254 for example) to the C_{H} portion of an IgG1. These two constructs, along with the ULC sequences (SEQ ID NO: 275 for example), are transfected into CHO cells and are co-expressed. The resulting antibodies are purified by a protein A column, followed by affinity purification with the appropriate target antigen (IL-4R or IL-2Rgamma) and the appropriate cell-specific antigen (Her2 or PSMA).

To demonstrate cell-specific heterodimerization of IL-4R and IL-2Rgamma receptors, a STAT3-luciferase assay is utilized. Ramos.2G5.4C10 cells are co-transfected with IL-4R, IL-2Rgamma, Her2 or PSMA, and STAT3-luciferase. For both Her2 and PSMA, cell surface-specific expression is selected for and receptor number is determined. Upon the cell-specific formation of IL-4R and IL-2Rgamma heterodimer in the presence of an IL-4 mimetic, luciferase will be transcribed, and a signal will be detected.

In the presence of an IL-4 mimetic (as disclosed in e.g., Domingues et al., 1999), activation is assessed using two bispecific Abs with target antigen binding domains that each bind to epitopes on IL4-R (e.g., T1 as shown in FIG. 1C) and IL2Rgamma (e.g., T2 as shown in FIG. 1C). The two bispecific mAbs will have two different cell-specific antigen binding domains that each bind to different epitopes on Her2 (e.g., different C1 and C2). In these experiments, non-blocking mAbs may be required in order to allow for IL4-R and IL2Rgamma on the cell surface. The combination of antibodies is incubated with the luciferase cell line above. It is expected that this combinations of the bispecific antibodies will be able to promote the formation of IL4R and IL-2Rgamma heterodimers and activate these receptors (i.e., activate the transcription of luciferase).

In the presence of an IL-4 mimetic (as disclosed in e.g., Domingues et al., 1999), activation is assessed using two bispecific Abs with target antigen binding domains that each bind to epitopes on IL4-R (e.g., T1 as shown in FIG. 1C) and IL2Rgamma (e.g., T2 as shown in FIG. 1C). The two bispecific mAbs will have two different cell-specific antigen binding domains that each bind to different or same/overlapping epitopes on PSMA (e.g., different or same C1 and C2). In these experiments, non-blocking mAbs may be required in order to allow for IL4-R and IL2Rgamma on the cell surface. The combination of antibodies is incubated with the luciferase cell line above. It is expected that both combinations of the bispecific antibodies will be able to promote the formation of IL4R and IL-2Rgamma heterodimers and activate these receptors (i.e., activate the transcription of luciferase).

The cell-specific activation of IL4R and IL-2Rgamma receptors may be useful in cancer treatments, e.g., breast cancer and prostate cancer treatments.

### Example 8

### Cell-Specific, Ligand-Dependent, Heterologous Activation of IFNAR1 and 2

IFNAR1 and IFNAR2 antibody sequences are derived from IFNAR1 and IFNAR2 scFv sequences (SEQ ID NOs 294-295, 298-300, 304-305, and 308-310, shown in Table 30 below).

TABLE 30

| SEQ | | | Nucleic acid/ |
|---|---|---|---|
| ID No. | Antibody fragment | Organism | polypeptide |
| 294 | anti-IFNAR-1_VH | Artificial sequence | Nucleic acid |
| 295 | anti-IFNAR-1_VH | Artificial sequence | Polypeptide |
| 298 | anti-IFNAR-1_HCDR1 | Artificial sequence | Polypeptide |
| 299 | anti-IFNAR-1_HCDR2 | Artificial sequence | Polypeptide |
| 300 | anti-IFNAR-1_HCDR3 | Artificial sequence | Polypeptide |
| 304 | anti-IFNAR-1_VH | Artificial sequence | Nucleic acid |
| 305 | anti-IFNAR-1_VH | Artificial sequence | Polypeptide |
| 308 | anti-IFNAR-1_HCDR1 | Artificial sequence | Polypeptide |
| 309 | anti-IFNAR-1_HCDR2 | Artificial sequence | Polypeptide |
| 310 | anti-IFNAR-1_HCDR3 | Artificial sequence | Polypeptide |

Her2 and PSMA antibody sequences are derived from ULC sequences and heavy chain sequences as discussed above.

To demonstrate target cell-specific clustering of IFNAR1 and IFNAR2, a Daudi cell assay is utilized. Daudi cells are co-transfected with IFNAR1, IFNAR2, Her2 or PSMA, and a detection agent. For both Her2 and PSMA, cell surface-specific expression is selected for and cell surface density is determined. IFNAR1 and IFNAR2 are thought to exist primarily as a heterodimer on the cell surface.

The bispecific antibodies are made recombinantly. In the first construct, the nucleic acid encoding the V_{H} portion of an anti-IFNAR1 antibody (SEQ ID NO: 294 for example) or an IFNAR2 antibody is fused to the C_{H} portion of an IgG1. A second construct is similarly made by fusing the nucleic acid encoding the V_{H} portion of an anti-Her2 antibody (SEQ ID NO: 238 for example) or an anti-PSMA antibody (SEQ ID NO: 254 for example) to the C_{H} portion of an IgG1. These two constructs, along with the ULC sequences (SEQ ID NO: 275 for example), are transfected into CHO cells and are co-expressed. The resulting antibodies are purified by a protein A column, followed by affinity purification with the appropriate target antigen (IFNAR1 or IFNAR2) and the appropriate cell-specific antigen (Her2 or PSMA).

In the presence of an IFN mimetic (as disclosed in, e.g., Ahmed et al., 2007), activation is assessed using two bispecific Abs with target antigen binding domains that each bind to epitopes on IFNAR1 (e.g., T1 as shown in FIG. 1C) and IFNAR2 (e.g., T2 as shown in FIG. 1C). The two bispecific mAbs will have two different cell-specific antigen binding domains that each bind to different epitopes on Her2 (e.g., different C1 and C2). In these experiments, non-blocking mAbs may be required in order to allow for IFNAR1 and IFNAR2 on the cell surface. The combination of antibodies is incubated with the Daudi cell line above. It is expected that this combination of the bispecific antibodies will be able to promote the formation of IFNAR1 and IFNAR2 heterodimers and activate the receptors (i.e., activate the transcription of luciferase).

In the presence of an IFN mimetic (as disclosed in, e.g., Ahmed et al., 2007), activation is assessed using two bispecific Abs with target antigen binding domains that each bind to epitopes on IFNAR1 (e.g., T1 as shown in FIG. 1C) and IFNAR2 (e.g., T2 as shown in FIG. 1C). The two bispecific mAbs will have two different cell-specific antigen binding domains that each bind to different or same/overlapping epitopes on PSMA (e.g., different or same C1 and C2). In these experiments, non-blocking mAbs may be required in order to allow for IFNAR1 and IFNAR2 on the cell surface. The combination of antibodies is incubated with the Daudi cell line above. It is expected that both combinations of the bispecific antibodies will be able to promote the formation of IFNAR1 and IFNAR2 heterodimers and activate the receptors (i.e., activate the transcription of luciferase).

The cell-specific activation of IFNAR1 and IFNAR2 receptors may be useful in the treatment of Hepatitis C, Chronic Myelogenous Leukemia, Renal Cell Carcinoma and Multiple Sclerosis.

### DOCUMENTS

AHMED, Chulbul M., et al. “IFN mimetic as a therapeutic for lethal vaccinia virus infection: possible effects on innate and adaptive immune responses.” The Journal of Immunology 178.7 (2007): 4576-4583.
ALTSCHUL, S. F., et al. (1990). Basic local alignment search tool. J Mol Biol. 215(3): 403-410.
ALTSCHUL, S. F., et al. (1997). Gapped BLAST and PSI-BLAST: a new generation of protein database search programs. Nucl. Acids Res. 25(17): 3389-3402.
CARDARELLI, J. M., et al. Interferon alpha receptor I antibodies and their use. U.S. Pat. No. 8,460,668 B2.
CHEN, L., et al. (2013). Molecular mechanisms of T cell co-stimulation and co-inhibition. Nature Reviews Immunology 13: 227-242.
DONG, H., et al. (1999). B7-H1, a third member of the B7 family, co-stimulates T-cell proliferation and interleukin-10 secretion. Nat Med 5(12): 1365-9.
DOMINGUES, Helena, et al. “Rational design of a GCN4-derived mimetic of interleukin-4.” Nature Structural & Molecular Biology 6.7 (1999): 652-656.
FACHINI, R., et al. Human fgf receptor and beta-klotho binding proteins. International Application Publication No. WO 2011/130417.
FOLTZ, Ian N., et al. “Treating diabetes and obesity with an FGF21-mimetic antibody activating the βKlotho/FGFR1c receptor complex.” Science translational medicine 4.162 (2012): 162ra153-162ra153.
GONNET, G. H., et al. (1992). Exhaustive matching of the entire protein sequence database. Science 256(5062): 1443-1445.
GUPTA, V. K., et al. (2013). TrkB Receptor Signalling: Implications in Neurodegenerative, Psychiatric and Proliferative Disorders. Int J Mol Sci 14(5): 10122-10142.
HASTEDT, J. E., et al. Storage stable power compositions of interleukin-4 receptor. U.S. Pat. No. 7,449,201 B2.
H U, S-F., et al. Human antigen binding proteins that bind beta-klotho, fgf receptors and complexes thereof. U.S. Patent Application No. 2011/0135657.
KAZANE, S. A., et al. (2013). Self-assembled antibody multimers through peptide nucleic acid conjugation. J Am Chem Soc. 135(1): 340-6.
KHARITONENKOV, A., et al. (2005). FGF-21 as a novel metabolic regulator. J Clin Invest 115: 1627-1635.
KHARITONENKOV, A., et al. (2007). The Metabolic State of Diabetic Monkeys Is Regulated by Fibroblast Growth Factor-21. Endocrinol. 148: 774-781.
KLEIN, C., et al. (2012). Progress in overcoming the chain association issue in bispecific heterodimeric IgG antibodies. mAbs 4(6): 653-663.
KÖHLER, G. and MILSTEIN, C. (1975). Continuous cultures of fused cells secreting antibody of predefined specificity. Nature 256: 495-497.
KONTERMANN, R. E., et al. (2011). Bispecific antibodies: developments and current perspectives. In: Bispecific antibodies. Kontermann, R. E. (ed.), Springer Heidelberg Dordrecht London New York, 1-28.
KOTH, C. M., et al. (2012). Molecular basis for negative regulation of the glucagon receptor. PNAS 109(36): 14393-8.
KUFER, P., et al. (2004). A revival of bispecific antibodies. Trends Biotechnol 22(5): 238-244.
LONG, Y. C., et al. (2011). Hormone-like fibroblast growth factors and metabolic regulation. Biochim Biophys Acta 1812(7): 791-5.
MASSA, Stephen M., et al. “Small molecule BDNF mimetics activate TrkB signaling and prevent neuronal degeneration in rodents.” The Journal of clinical investigation 120.5 (2010): 1774.
MULLER, D. and KONTERMANN, R. E. (2011). Diabodies, single chain diabodies and their derivatives. In: Bispecific antibodies. Kontermann, R. E. (ed.), Springer Heidelberg Dordrecht London New York, 83-100.
NAKAHASHI-ODA, C., et al. (2012). Apoptotic cells suppress mast cell inflammatory responses via the CD300a immunoreceptor. JEM 209(8): 1493-1503.
OGAWA, Y., et al. (2007). Beta-Klotho is required for metabolic activity of fibroblast growth factor 21. PNAS 104(18): 7432-7437.
PADLAN, E. A., et al. (1995). Identification of specificity-determining residues in antibodies. FASEB J. 9: 133-139.
RIBAS, A., et al. (2012). Tumor immunotherapy directed at PD-1. NEJM 366(26): 2517-9.
RILEY, J. L., et al. (2009). PD-1 signaling in primary T cells. Immunol Rev. 229:114-125.
SANAK, M., et al. (2007). Genetic Variability of the High-affinity IgE Receptor α Subunit (FεERIα) is Related to Total Serum IgE levels in Allergic Subjects. Allergology International 56: 397-401.
SCATCHARD, G. (1949). The attractions of proteins for small molecules and ions. Ann NY Acad Sci 51: 660-72.
SLEEMAN, M. W., et al. (2000). The ciliary neurotrophic factor and its receptor, CNTFRa. Pharmacochemistry Library 31: 265-272.
SONODA, J., et al. Fgfr1 agonists and methods of use. U.S. Patent Application No. 2012/0294861.
STEVENS, S., et al. High affinity antibodies to human IL-6 receptor. U.S. Patent Application No. 2007/0280945 A1.
SUZUKI, M., et al. (2008). Beta-Klotho Is Required for Fibroblast Growth Factor (FGF) 21 Signaling through FGF Receptor (FGFR) 1c and FGFR3c. Mol Endocrinol 22: 1006-1014.
TANG, Zhongshu, et al. “An optic nerve crush injury murine model to study retinal ganglion cell survival.” Journal of visualized experiments: JoVE 50 (2011).
TUTT, A., et al. (1991). Trispecific F(ab′)3 derivatives that use cooperative signaling via the TCR/CD3 complex and CD2 to activate and redirect resting cytotoxic T cells. J Immunol. 147(1): 60-69.
VAJDOS, F. F., et al. (2002). Comprehensive Functional Maps of the Antigen-binding Site of an Anti-ErbB2 Antibody Obtained with Shotgun Scanning Mutagenesis. J Mol Biol 320(2): 415-428.
WANG, X. F., et al. (2013). PD-1/PDL1 and CD28/CD80 pathways modulate natural killer T cell function to inhibit hepatitis B virus replication. Journal of Viral Hepatitis 20: 27-39.
WATANABE, N., et al. (2012). Coinhibitory Molecules in Autoimmune Diseases. Clin Dev Immunol 2012:269756.
YAO, V., et al. (2009). Expression of prostate-specific membrane antigen (PSMA), increases cell folate uptake and proliferation and suggests a novel role for PSMA in the uptake of the non-polyglutamated folate, folic acid. Prostate 70(3): 305-16.
YARDEN, Y. (2001). Biology of HER2 and its importance in breast cancer. Oncology 61(Suppl 2): 1-13.

Although illustrative embodiments of the present invention have been described herein, it should be understood that the invention is not limited to those described, and that various other changes or modifications may be made by one skilled in the art without departing from the scope or spirit of the invention.

What is claimed is:

1. A method for cell-specific modulation of a target antigen comprising contacting a target cell having the target antigen on the surface of the target cell with:
a. a first multi-specific antigen-binding polypeptide comprising:
(i) a cell-specific antigen binding domain (C1) and
(ii) a target antigen binding domain (T1); and
b. a second multi-specific antigen-binding polypeptide comprising:
(i) a cell-specific antigen binding domain (C2) and
(ii) a target antigen binding domain (T2);
wherein C1 and C2 interact with the same cell-specific antigen, and the cell-specific antigen and the target antigen are on the same target cell.

2. The method according to claim 1, wherein C1 and C2 bind different epitopes on the cell-specific antigen.

3. The method according to claim 1, wherein T1 and T2 bind the same epitope on the target antigen.

4. The method according to claim 1, wherein the cell-specific antigen is a monomer on the cell surface.

5. The method according to claim 1, wherein the cell-specific antigen is composed of at least two polypeptide subunits on the cell surface.

6. The method according to claim 5, wherein the cell-specific antigen is a homodimer on the cell surface.

7. The method according to claim 1, wherein the target antigen is composed of at least two copies of the same polypeptide subunit on the cell surface.

8. The method according to claim 7, wherein the target antigen is a homodimer on the cell surface.

9. The method according to claim 1, wherein the target antigen is composed of at least two different polypeptide subunits on the cell surface.

10. The method according to claim 9, wherein the target antigen is a heterodimer on the cell surface.

11. The method according to claim 9, wherein the target antigen is a heterotrimer on the cell surface.

12. The method according to claim 1, wherein the first multi-specific antigen-binding polypeptide(s) further comprise(s):
(iii) a first multimerizing domain (M1) and optionally a second multimerizing domain (M2);
and the second multi-specific antigen-binding polypeptide further comprises
(iv) a first multimerizing domain (M3) and optionally a second multimerizing domain (M4).

13. The method according to claim 12, wherein at least one of M1, M2, M3 or M4 is a polypeptide comprising an immunoglobulin C_{H}2 domain or an immunoglobulin C_{H}3 domain.

14. The method according to claim 13, wherein at least one of M1, M2, M3 or M4 comprises an Fc domain of an immunoglobulin.

15. The method according to claim 1, wherein at least one of C1, C2, T1 or T2 comprises an epitope-binding domain selected from the group consisting of: (i) a Fab; (ii) an scFv; (iii) a dAb; (iv) a V_{H}/C_{H}1; (v) a V_{L}/C_{L}; and (vi) a domain antibody.

16. The method according to claim 15, wherein the epitope-binding domain is a Fab or an scFv.

17. The method according to claim 1, wherein at least one of the first and second multi-specific antigen-binding polypeptides is a bispecific antibody.

18. The method according to claim 17, wherein one of the antigen binding domains of at least one bispecific antibody has at least a two fold lower affinity for its target relative to the other antigen binding domain in the same bispecific antibody.

19. The method according to claim 1, wherein the cell surface density of the cell-specific antigen is lower than the cell surface density of the target antigen.

20. The method according to claim 1, wherein the first and second multi-specific antigen-binding polypeptides are present in excess relative to the target antigen.

21. The method according to claim 1, wherein the target antigen is a receptor and modulation is activation of the receptor.

22. The method according to claim 21, wherein activation of the receptor is ligand dependent.

23. The method according to claim 22, wherein activation of the receptor is ligand independent.

24. The method according to claim 1, wherein the target antigen is a receptor and modulation is inhibition of the receptor.

25. The method according to claim 1, wherein the cell-specific antigen and the target antigen form a complex on the surface of the cell.

26. The method according to claim 1, wherein at least one of C1 and C2 comprises a ligand or portion of a receptor that specifically binds the cell-specific antigen.

27. The method according to claim 1, wherein at least one of T1 and T2 comprises a ligand or portion of a receptor that specifically binds the target antigen.

28. A pharmaceutical composition for cell-specific modulation of a target antigen in a subject in need thereof, the pharmaceutical composition comprising a pharmaceutically acceptable diluent or carrier and an effective amount of:
a. a first multi-specific antigen-binding polypeptide comprising:
(i) a cell-specific antigen binding domain (C1) and
(ii) a target antigen binding domain (T1); and
b. a second multi-specific antigen-binding polypeptide comprising:
(i) a cell-specific antigen binding domain (C2) and
(ii) a target antigen binding domain (T2),
wherein C1 and C2 interact with the same cell-specific antigen and the cell-specific antigen and the target antigen are on the same target cell.

29. The pharmaceutical composition according to claim 28, wherein the subject is a mammal.

30. The pharmaceutical composition according to claim 29, wherein the mammal is a human.

31. The pharmaceutical composition according to claim 30, which is in a unit dosage form comprising both multi-specific antigen-binding polypeptides.

32. The pharmaceutical composition according to claim 31 in which the first multi-specific antigen-binding polypeptide is in a first unit dosage form and the second multi-specific antigen-binding polypeptide is in a second unit dosage form, separate from the first.

33. A kit for cell-specific modulation of a target antigen comprising, packaged together with instructions for their use:
a. a first multi-specific antigen-binding polypeptide comprising:
(i) a cell-specific antigen binding domain (C1) and
(ii) a target antigen binding domain (T1); and
b. a second multi-specific antigen-binding polypeptide comprising:
(i) a cell-specific antigen binding domain (C2) and
(ii) a target antigen binding domain (T2),
wherein C1 and C2 interact with the same cell-specific antigen, and the cell-specific antigen and the target antigen are on the same target cell.

34. A method for cell-specific modulation of a target antigen comprising contacting a target cell having the target antigen on the surface of the target cell with:
a. a first multi-specific antigen-binding polypeptide comprising:
(i) a cell-specific antigen binding domain (C1) and
(ii) a target antigen binding domain (T1);
b. a second multi-specific antigen-binding polypeptide comprising:
(i) a cell-specific antigen binding domain (C2) and
(ii) a target antigen binding domain (T2); and
c. an antigen-binding polypeptide comprising:
(i) a first cell-specific antigen binding domain (C3) and
(ii) a second cell-specific antigen binding domain (C4),
wherein the cell-specific antigens and the target antigen are on the same target cell, C1 and C3 interact with a first cell-specific antigen, and C2 and C4 interact with a second cell-specific antigen.
